(19) United States
(12) Patent Application Publication
(10) Pub. No.: US 2024/0052288 A1
(43) Pub. Date: Feb. 15, 2024

(54) IMAGE ANALYSIS AND NON-INVASIVE DATA COLLECTION FROM CELL CULTURE DEVICES

(71) Applicant: CelVivo ApS, Blommenslyst (DK)

(72) Inventors: Stephen John FEY, Blommenslyst (DK); Hans Henrik JOCHUMSEN, Blommenslyst (DK); Krzysztof WRZESINSKI, Blommenslyst (DK)

(21) Appl. No.: 18/260,429

(22) PCT Filed: Jan. 4, 2022
(86) PCT No.: PCT/EP2022/050072

§ 371 Date: Jul. 5, 2023

(30) Foreign Application Priority Data
Jan. 4, 2021 (DK) PA202170002

(51) Int. Cl. C12M 1/36; C12M 3/04; C12M 1/34; G06T 7/11; G06T 7/00; G06V 20/52; G06T 7/73; G06V 20/69

(52) U.S. Cl. CPC C12M 41/48; C12M 27/10; C12M 41/42; G06T 7/11; G06T 7/0012; G06V 20/52; G06T 7/73; G06V 20/698; G06V 20/693; G06T 2207/30024; G06T 2207/10016; G06T 2207/20021; G06T 2207/30204

(57) ABSTRACT

An incubator system configured to illuminate at least one or more cell culture chamber devices and to rotate them about a respective predetermined axis, each cell culture chamber device comprising an enclosure, the enclosure configured to contain a cell culture media, and at least one viewing area configured to allow inspection of at least a part of the cell culture media, the incubator system further comprising at least one illumination device and at least one monitoring device, the at least one monitoring device configured to provide one or more monitoring signals of at least part of an illuminated cell culture media or an illuminated cell culture chamber, illuminated, by at least one of the at least one illumination device, with a signal of electromagnetic radiation, preferably incoherent or coherent ultraviolet, visible, infrared, and/or near-infrared light of broad or narrow wavelength spectrum, and wherein the incubator system further comprises or is in connection with one or more processing units configured to extract and/or derive data from the one or more monitoring signals, said data representing one or more aspects of the cell culture chamber device itself and/or the cellular activity occurring within.

### FIELD OF THE INVENTION

The present invention relates generally to extracting and/or deriving data from images of cell culture chamber devices (also equally referred to herein as bioreactors) where each cell culture chamber device comprises an enclosure (also equally referred to herein as a cell chamber) configured to contain a cell culture media (typically also comprising cells), to interpret at least some of the data in real or near-real time and without operator-introduced bias, and to use some of this data to, for example, adjust the operation of the incubator system which regulates the environment for the cells.

### BACKGROUND

When growing cells and tissue using more traditional cell culture chamber devices, often having an essentially flat cell support surface or the like, primary cells and biopsies tend to de-differentiate and lose their normal structural organisation and in vivo functionality. One example of this is where cells migrate from a block of tissue out onto the flat supporting surface (i.e. the so-called “melting ice-cream effect”). De-differentiated cells typically express different biochemical properties than those normally expressed by corresponding mature cells in tissues in an intact organism. Furthermore, certain cells have typically lost their specialised functions compared to corresponding cells in an intact organism. Even cancer cells are forced to grow faster than they do in the organism when they are cultured on flat surfaces. In doing so, the cancer cells are prevented from expressing a closer-to-normal phenotype and are thus not a good model for cancer in vivo.

Improving on this, certain cell culture chamber devices or bioreactors for the growing of cells, whether of single or several cell types, or tissues, normally or even preferably use operation under omnidirectional normogravity (also called simulated microgravity) conditions i.e. clinostat induced conditions, since this enables the preservation of the differentiated state of many types of cell in the culture. Furthermore, it promotes the recovery (or re-differentiation) of in vivo like structure and functionality in many different cell lines. This is significant because cell lines are used for the majority of cell culture work currently executed. The same is the case for the rapidly growing field of primary cells or stem cells (whether native or induced in any way): they all retain or obtain some degree of in vivo like structure and functionality.

Such simulated microgravity may be induced by continuous rotation of a compartment containing the cell culture (in one, two, or three dimensions), thereby preventing the cells to adhere to the compartment walls. Strictly speaking, the rotation infinitesimally increases the gravitational force (centripetal acceleration) but since gravity is applied from all sides, the net gravitational effect is close to null. Suitable rotation promotes the adherence of cells to each other in a fluid environment with a minimum of shear forces acting on the culture. Shear forces can be introduced, if needed, for specific cell/tissue types, by changing the rotational speed of the cell culture chamber device (see Kraus et al., 2020, doi: 10.1016/j.mvr.2020.104107. Thereby, cells aggregate into colonies in 3D, typically named clusters, aggregates, spheroids, organoids, prototissues, or pseudotissues (in this disclosure referred to collectively as spheroids). Since pieces of extirpated tissue will be affected similarly, they are also included under the generic term ‘spheroids’.

At least for certain incubators, several cell culture chamber devices or bioreactors, e.g. with different types and/or sizes/state of cells, are used in the incubator where they all typically are located in the same—closable—open space or cavity. Even if provided with internal lighting, use in an incubator will reduce individual visibility of the content of each cell culture chamber device, often prompting users to repeatedly open and close—over time—the incubator and optionally take out a cell culture chamber device for closer manual inspection and processing. Repeatedly, opening and closing the incubator may at least increase the risk of contamination and at least temporarily disrupt the controlled internal environment of the incubator. More specifically, removal of a cell culture chamber device from the incubator to, for example, a sterile bench or an imaging microscope potentially exposes the cells to a very low CO₂ level (atmospheric CO₂ is ca. 0.04%), a high O₂ level (ca. 21%), ambient temperature (probably around 22° C. in a laboratory), excess light and normogravity (1G).

Removing a cell culture chamber device from the incubator for inspection will typically cause the partial pressure of CO₂ and temperature both around and inside the cell culture chamber to fall. A fall in the partial pressure of CO₂ will cause the pH of the growth media to become more basic and this will affect cellular metabolism. Sudden changes in temperature (by only a few degrees and for only a few minutes)) have been shown to induce changes in expression of the genetic information and result in the synthesis of so called ‘heat-shock’ (heat-stress) or ‘cold-shock’ response. Several of the ‘molecular chaperone’ proteins induced by temperature changes are involved in assisting proteins to fold in the correct conformation or in the proteasomal degradation of mis-folded proteins. But the response goes much further than these few proteins (see e.g. Richter et al. 2010 doi: 10.1016/j.molcel.2010.10.006). In certain known types of incubators, it has for example been shown that after opening the door to the incubation chamber for only about 30 seconds it took about 6 minutes to re-establish the proper temperature and CO₂ level again.

Removing a cell culture chamber device from the incubator for inspection will typically also cause the cell clusters or spheroids to sink to the bottom. Here they will typically experience reduced gas exchange (localised decrease in the partial pressure of oxygen and an increase in the partial pressure of CO₂), a fall in nutrient levels and a build-up of waste metabolites which could lead to cell death by necrosis.

Thus, removing a cell culture chamber device from the incubator for inspection will typically cause the partial pressure of O₂ to fall. Oxygen and its free radicals (including superoxide anion radical (O₂⁻), singlet oxygen (¹O₂), hydroxyl radical (·OH) and perhydroxyl radical (HO₂·), termed collectively the ‘reactive oxygen species’ (ROS)) are highly reactive and can damage the majority of biological molecules. This oxidation damage has been suggested to be involved in many processes including carcinogenesis, tumourigenesis and ageing. Cells in the cell culture chamber device will experience a surge of damaging ROS as oxygen returns when the device is returned to the incubator system.

Removing a cell culture chamber device from the incubator for inspection will also cause changes in the gravitation and the shear forces experienced by the cells or spheroids, both of which can also affect the expression of the genetic information (see Penthó et al., 2019 doi: 10.1016/j.ceca.2019.03.007 and Marin et al. 2013 doi: 10.1016/j.freeradbiomed.2013.05.034).

When opening or accessing the incubation chamber, e.g. for inspection, the cell culture chamber devices and their enclosures are exposed to light, which potentially is detrimental for certain types of cells and spheroids.

Finally, removing a cell culture chamber device from the incubator for inspection and processing will also expose it to an increased risk of infection by microorganisms (e.g. viruses, bacteria, fungi) or even contamination (by for example mycoplasma or other cell types).

As the spheroids grow, they get bigger and thus the rate of rotation of the cell culture chamber device needs, at least for certain uses, to be adjusted to maintain optimal conditions where the spheroids remain in an essentially ‘stationary orbit’ relative to the cell culture chamber device as this promotes gas and nutrient exchange, improved uniformity of the spheroids and it reduces shear forces to a minimum. This is a preferred condition for certain cell types because the shear stress for the spheroids is minimised and the number of collisions, and the ‘force of impact’ between the spheroids or between spheroids and the walls of the cell culture chamber is also reduced. However, in any event it is very beneficial to be able to clearly inspect the spheroids in the cell culture chamber device at several occasions e.g. to see whether a speed adjustment should be made, and potentially to what extent. This task is normally carried out by opening the outer door of the incubator, which may, depending on the construction of the incubator affect the internal environment. However, this speed adjustment must be made many times each day during the early days of culture and then repetitively during the weeks or months of culture, and the degree of correction is subjective and depends on the user and how frequently the user inspects the culture. Thus, there is a need to improve visualisation of the spheroids in order to keep spheroids in a repeatedly optimised growth condition: this would lead to improved spheroid uniformity, removing the potential for subjective bias. Improved uniformity of the spheroids results in a more standardized metabolic performance which then enables for example a more reliable in vitro predictive toxicological evaluation of candidate drugs prognosis of the cell culture before going into expensive clinical trials or similar, i.e. it results in a more reliable “filter” prior to embarking on animal or clinical trials.

Furthermore to this, it is possible to harvest a great deal of additional data and information from the spheroids or the medium in which they grow, or even the cell culture chamber itself all of which can facilitate or accelerate the user's work without disturbing the culture and thus without exposing the culture to changes in temperature, gas partial pressures, the effects of gravity and in particular, the risk of infection.

For other uses, the spheroids need not or possibly also should not remain in a stationary orbit but rather be allowed a different behaviour e.g. be allowed to tumble against the cell culture chamber walls, be located on or near the bottom of the cell culture chamber, or be held against the wall of the cell culture chamber by centripetal acceleration, etc. In these situations, a minimisation of shear forces is not needed and potentially is disadvantageous for the cells to mimic in vivo performance.

Accordingly, it would be a benefit to provide an incubator which facilitates the inspection of the cultures within, and which in addition reduces the need for opening and closing the incubator. It would also be a benefit to apply rotational speed corrections as the spheroids change their size. By tracking individual spheroids, it would be possible to calculate their rate of fall in the culture. This information could be used to optimise the rotational speed of the cell culture chamber.

Accordingly, it would be a benefit to provide an incubator facilitating the collection of additional data and/or information from the cell culture in a non-invasive, e.g. on the fly, basis. It would be a further benefit if this data/information could be used to modify or set or control the operation of the incubator itself or provide the user with data that would otherwise be tedious, difficult, or practically impossible to collect.

Accordingly, to all of the above, automation and feedback regulation of the incubator would lead to a further standardisation of operation and quality of the data obtained by further reducing subjective user intervention. From an economic viewpoint, the cost of operating the incubator may also be reduced.

Accordingly, it would be an advantage to provide an incubator addressing one or more of the above-mentioned drawbacks, at least to an extent. In particular, it would be a further or additional advantage to provide an incubator system that could process or interpret data collected in a way which would reduce the need for opening and closing it. It would be a further or additional advantage to provide an incubator enabling data processing which would allow the operator to reach decisions in an expedited and timely manner and which could provide early indications of an experiment that is not developing in the expected manner or that the experiment has finished (for example if following treatment, the cells in the spheroids are dead) thus releasing the equipment for another experiment.

### SUMMARY

It is an object to provide an incubator system addressing one or more of the above-mentioned drawbacks, at least to an extent. It is a further object to provide an incubator system providing enhanced viewing of the content of any contained cell culture chamber devices and/or their respective enclosures (i.e. the cell culture media contained therein). It is yet another object to provide an incubator system configured so that obtained data, e.g. obtained images, can be electronically processed to extract and/or derive (further) data therefrom. It is a fourth object (at least in some embodiments) to provide an incubator system which can issue alerts to a user or another device or system when predefined threshold(s) relating to the growth of cells is/are exceeded. Finally, it is an object (at least in some objects) to provide an incubator system which can use some of the data extracted or derived from the images to modify its own function.

According to a first aspect, one or more of these objectives is achieved, at least to an extent, by an incubator system configured to receive a predetermined number, at least one or a plurality, of cell culture chamber devices. The predetermined number of cell culture chamber devices may e.g. be 1, 2, 3, 4, 5, 6, or more. The incubator system is furthermore configured to receive, rotate (e.g. or preferably individually) about a respective predetermined axis, and illuminate a predetermined number (at least one or a plurality) of cell culture chamber devices, each cell culture chamber device comprising an enclosure, the enclosure configured to contain a cell culture media, where the cell culture chamber device comprises at least one viewing end or part configured to allow for inspection of at least a part of content of the enclosure, wherein the incubator system further comprises at least one illumination device and at least one monitoring device, the at least one monitoring device configured to detect, monitor, or register at least one illuminated received cell culture chamber device wherein the at least one monitoring device can be an imaging or vision system or device and the monitoring signal comprises electromagnetic radiation, preferably incoherent or coherent ultraviolet, visible, infrared, and/or near-infrared light of broad or narrow wavelength spectrum, and wherein the incubator system is configured to capture one or more images, and/or other type of monitoring signal(s), of the cell culture chamber device, and wherein the incubator system further comprises one or more processing units configured to extract and/or derive data from at least one, some, or all of the images and/or other type(s) of monitoring signal(s). In at least some embodiments or in at least some preferred embodiments, each respective predetermined axis that a cell culture chamber device rotates about (or is able to rotate about) is a horizontal or a substantially horizontal axis.

In some embodiments, the incubator system is an incubator as described in one or more embodiments of applicant's co-pending PCT patent application titled “AN INCUBATOR FOR RECEIVING A NUMBER OF CELL CULTURE CHAMBER DEVICES” with application number PCT/EP2021/067777, incorporated herein in its entirety by reference.

In some embodiments, the cell culture chamber device is a cell culture chamber device as described in one or more embodiments of applicant's co-pending PCT patent application titled “A CELL CULTURE CHAMBER DEVICE FOR CELL AND TISSUE GROWTH” with application number PCT/EP2021/064742, incorporated herein in its entirety by reference.

In some embodiments, the incubator system
comprises a housing comprising an incubation chamber configured to contain at least a respective part of the cell culture chamber devices when received by the incubator, and cell culture chamber device,
is configured to rotate a received cell culture chamber device about (at least) a predetermined rotational axis, e.g. or preferably a horizontal axis, of the received cell culture chamber device,
comprises at least one illumination device being aligned with or integrated with the incubator and being configured to illuminate at least part of one cell culture chamber device for at least short periods of time from at least one and potentially more sides,
comprises at least one monitoring device being aligned with or integrated with the incubator and being configured to register and/or detect an monitoring signal after passing, reflecting, or propagating through at least a part of the enclosure of at least one of the cell culture chamber devices when received by the incubator,
comprises an element to capture one or more single images, optionally from a video sequence, for example by using a frame-grabber (which can either be hard or software based) and which may or may not be an integral part of the incubator system), and/or
is configured to provide obtained images to one or more image processing devices, e.g. running during use one or more image processing programs, which may or may not be an integral part of the incubator system.

In some embodiments, a cell culture chamber device (also equally referred to herein as a bioreactor) as disclosed herein comprises an enclosure (or cell chamber) configured to contain a typically aqueous cell culture media and usually cells. The enclosure would be defined by a first end, a second end, and at least one connecting (e.g. circumferential) wall connecting the first and the second ends. The first end may be referred to as an illumination end or part, or as a primary or front-illumination end or part. The first end may e.g. also be referred to as a viewing end or part, or as a primary viewing end or part. The first end, or a part or window thereof, is substantially transparent. The second end and/or the connecting wall, or a respective part or window thereof, is/are substantially transparent or is substantially translucent. The first end or the part or window thereof is configured to be optically or otherwise (e.g. or i.e. with respect to other electromagnetic radiation or mechanical waves such as sound or acoustic waves) aligned (at least for some period of time or periodically) with the second end or the part or window thereof and/or the with the connecting wall or the part or window thereof so that light or another monitoring signal, can be transmitted through or by the second end or the part or window thereof and/or through or by the connecting wall or the part of window thereof into the enclosure, is transmitted or propagates through at least a part of the cell culture media and out through the first end or the part or window thereof to outside the enclosure, and e.g. to outside of the cell culture chamber device. The first end (or the part or window thereof) does not need to be optically or otherwise aligned with the second end (or the part or window thereof) by being across or directly across each other, even though that provides a very expedient way of providing this. For example, a suitable optically based or other electromagnetically radiation based, sound/acoustic wave based, etc. system or one or more suitable devices or components (e.g. reflectors, mirrors, lenses, prisms, sound or light-guides, etc.) could be used to align the respective ends (or parts/windows) at least during some time.

A cell culture chamber device may further comprise one or more fiducial and/or identification markers, such as identification markings, barcodes, points of reference, etc. At least some of the fiducial and/or identification markers is/are preferably machine readable. The cell culture chamber device may also be constructed with other features such as access ports, whether axial, radial or circumferential (for example, for the changing of media, collection of samples, injection of compounds etc.), gas exchange membranes (for the exchange of for example O₂, CO₂) and vents, humidification devices, markers or indicators. In principle, the cell culture chamber device might have any suitable regular or irregular shape (while supporting rotation as described herein) but it is preferred for manufacturing purposes if the shape is relatively simple. It is clearly beneficial if these additional parts do not obstruct illumination or inspection of the enclosure at least in part or as a whole for at least as little or preferably no time. Furthermore, it is beneficial that these additional parts cause as little or preferably no interference with the illumination or inspection of the enclosure.

Accordingly, an incubator is described which provides an un-obstructed light or other monitoring signal propagation path propagating through at least a part of any cell culture media and cells contained in the enclosure of a cell culture chamber device for at least part of the time. It also enables the provision of back-light or emission of another monitoring signal from ‘behind’, i.e. light shone or another monitoring signal emitted through the second end and/or the connecting wall(s) (e.g. towards the second end), greatly enhancing visual inspection (manual or automatic) from the other/opposite side (i.e. via the first end). This is particularly useful e.g. for inspection of several cell culture chamber devices arranged in the incubator or the like. If the second end of the cell culture device is transparent, then visual or other inspection (e.g. acoustic or electromagnetic radiation different from light), manual (in case of light) and/or automatic (in case of light or other electromagnetic radiation or sound or acoustic waves using a suitable sensor), is furthermore enabled from both ends, i.e. two sides, of the enclosure. In some embodiments, all the parts of the cell culture chamber device are transparent.

Such a cell culture chamber device is furthermore especially useful for use in connection with the incubator according to the first aspect and as disclosed herein, since the transparent first end allows for increased quality of detection and/or registration of its content by one or more one monitoring device(s) as disclosed herein.

As mentioned, according to the first aspect and as disclosed herein, one or more of such cell culture chamber device(s) can be mounted in a suitably configured incubator.

In some embodiments, the second end or the part or window thereof of the cell culture chamber device is substantially transparent (instead of substantially translucent) and the cell culture chamber device further comprises or is connected to a light diffusor (also referred to as optical diffusor) of the incubator configured to receive light and to provide substantially uniform light to the second end or the part or window thereof thereby providing substantially uniform illumination of the cell culture media when contained in the enclosure. The light diffusor is located in the light propagation path between the light source (natural and/or artificial) and before the enclosure/the second end or the part or window thereof. The substantially uniform illumination of the cell culture media in this way readily enables enhanced visual (manual or automatic) monitoring and thereby visual assessment of the content of the enclosure while it is still in the incubator.

For alternative embodiments, where the second end or the part or window thereof is substantially translucent (instead of substantially transparent), the translucent end or part or window will effectively function as a light diffuser thereby saving the need for such an additional component. For further alternative embodiments, where the second end or the part or window thereof is substantially translucent (instead of substantially transparent), a light diffuser is still present, thereby in effect providing a double-diffusor (one by the translucent end or part or window thereof and one by the light diffuser) that may produce an even further uniform light distribution (at the ‘cost’ of some but typically not a lot of light energy).

The incubator thus comprises one or more light (or illumination) sources configured to illuminate at least a respective part of the cell culture chamber devices, e.g. illuminating at least a part of respective enclosure of any received cell culture chamber devices. In yet further embodiments, the one or more light (or illumination) sources is/are configured to respectively illuminate at least a first end, or a part or window thereof, of an enclosure of one or more cell culture chamber devices received by the incubator. In some further embodiments, the one or more light sources is/are arranged in the door or lid facing the one or more cell culture chamber device(s) when received by the incubator. Accordingly, (primarily) ‘front’-illumination of the enclosure(s) is/are provided, where ‘front’-illumination is to be taken as illumination originating from the opening of the incubation chamber toward the cell culture chamber device(s) (when received).

In some embodiments, at least one respective drive unit (e.g. one, some or all) of the at least one rotational drive unit comprises one or more light or illumination sources configured to illuminate at least a respective part of the cell culture chamber device(s), e.g. illuminating at least a part of respective enclosure of any received cell culture chamber device(s). In yet further embodiments, the one or more light or illumination sources is/are configured to respectively illuminate at least a second end, or a part or window thereof, of an enclosure of one or more cell culture chamber device(s) received by the respective drive unit.

In some alternative embodiments, the at least one respective drive unit (e.g. one, some or all) of the at least one rotational drive unit comprises a hollow rotational shaft comprising a light guide or other light or illumination device configured to illuminate at least a respective part of a cell culture chamber device or an enclosure of such when received by the drive unit.

Accordingly, (primarily) ‘back’-illumination of the enclosure(s) is/are provided, where ‘back’-illumination is to be taken as illumination originating opposite the opening of the incubation chamber and towards the cell culture chamber device(s) (when received).

In some embodiments, the at least one drive unit (or at least the ones providing back-lighting) comprises a cavity arranged adjacent to a received cell culture chamber device where the cavity comprises the one or more light or illumination sources. This provides a very compact drive unit with back-lighting.

In some embodiments, the one or more light or illumination sources are arranged offset from a central axis or rotational axis of the enclosure or of cell culture chamber device, which may provide simple/simpler access to the enclosure and thereby to its content.

Embodiments of the incubator may comprise a mix of front-illuminating and back-illuminating light or illumination sources, increasing the quality of any obtained monitoring signals. Embodiments may alternatively comprise only one or more back-illuminating light or illumination light sources or comprise only one or more front-illuminating light or illumination light sources.

The light is at least in some embodiments natural or artificial light or a combination thereof, typically or preferably visible light having a wavelength of about 400 to about 700 nanometres or at least a sub-range thereof. Alternatively, the light could e.g. be infrared or near-infrared light respectively having a wavelength of about 700 nanometres to about 1 millimetre or about 900 nanometres to about 2500 nanometres. As yet another alternative, the monitoring signal is an electromagnetic radiation having a wavelength different from visible light or light, e.g. an ultraviolet light having wavelengths of about 10 to 400 nanometres. By (substantially) transparent and (substantially) translucent is meant that the ends or walls (or respective parts or windows thereof) are sufficiently (substantially) transparent and/or sufficiently (substantially) translucent in relation to the type of light or other monitoring signal intended to be used with the cell culture chamber device. In specific embodiments, particular wavelengths can be selected by using filters or diffraction gratings or the light can be coherent as in a laser.

In yet further alternative embodiments, the diffusor is not a light diffuser but a diffusor with respect to the other type of monitoring signal, e.g. an acoustic diffusor or a diffusor for electromagnetic radiation other than light.

In some alternative embodiments, the cell culture chamber device is configured for front-lighting (or other front-application of the other type of monitoring signal) either in addition to or as an alternative to back-lighting or emission of another monitoring signal from ‘behind’.

In some embodiments, the second end and/or at least one of the at least one connecting wall comprises one or more integrated light sources.

In some embodiments, the second end and/or at least one of the at least one connecting wall is/are or comprises a fluorescent light emitting element.

In some alternative embodiments, the cell culture chamber device is configured for side-lighting (or other side-application of the other type of monitoring signals) either in addition to or as an alternative to back- or front-lighting or emission of another monitoring signal from ‘behind’ or the ‘side(s)’.

In some such further alternative embodiments, the diffusor (if one is present) may be replaced by a suitable reflector, e.g. a parabolic reflector.

The at least one illumination device can in principle (at least in some embodiments) be located outside the incubator instead of being integrated with it, where the incubator then comprises a number of transparent windows or the like aligned with the cell culture chamber device(s) (when received by the incubator) allowing the illumination device to illuminate the content of any received cell culture chamber device(s) when properly arranged. The illumination of the cell culture chamber device can be from the same side as the monitoring device (front-illumination) and in this configuration will rely primarily on reflected or reemitted light reaching the monitoring device. The illumination of the cell culture chamber device can be from the opposite side as the monitoring device (back-illumination) and in this configuration will rely on transmission, silhouette, phase differences, or reemitted light reaching the monitoring device. Side illumination of the cell culture chamber device will result in a combination of the two effects mentioned above.

According to the first aspect, in this way, monitoring (and/or other registration and/or detection) is readily enabled where the monitoring e.g. may be local or even remotely as further disclosed herein. The monitoring signal may e.g. be an electromagnetic (usually ultraviolet, visible, infrared, and/or near-infrared light) signal and the monitoring device(s) may e.g. be cameras, CCDs, photomultipliers, radio receivers or the like configured to providing data or a video feed or video capture, (e.g. periodic), still images, etc. of the content of the enclosure(s) of any contained cell culture chamber devices. Alternatively, the monitoring signal is a different signal, e.g. as disclosed herein.

The at least one monitoring device can in principle (in some embodiments) be located outside the incubator instead of being integrated with it, where the incubator then comprises a number of transparent windows or the like aligned with the cell culture chamber device(s) (when received by the incubator) allowing the monitoring devices to register and/or detect the content of any received cell culture chamber device(s) when properly arranged.

In some embodiments, the incubator comprises an openable and closable door or lid (or other access element) and the incubation chamber comprises at least one incubation chamber wall, the at least one incubation chamber wall and the door or lid (or other element), when closed, defining the incubation chamber at least in part. In some embodiments, the at least one incubation chamber wall and the door or lid (or other access element), when closed, defines the incubation chamber in full. In some embodiments, the incubation chamber comprises only a single incubation chamber wall

In some further embodiments, a first or an inner side (i.e. the side facing the incubation chamber when the door, lid, etc. is closed) of the door or lid (or other access element) comprises the at least one monitoring device where the at least one monitoring device is/are arranged facing so that an enclosure of at least one received cell culture chamber device is within a field of view of registration and/or detection of at least one of the at least one monitoring device, i.e. the enclosure(s) of any received cell culture chamber device(s) is within the field of view of at least one monitoring device.

In some embodiments (with a plurality of monitoring devices), the monitoring devices are arranged, e.g. equidistantly, in a substantially circular or other regular pattern in the door, lid, or the like of the incubator.

In some embodiments, the incubator comprises the predetermined number of monitoring devices, i.e. one monitoring device for each cell culture chamber device that can be received by the incubator or in other words in a one-to-one relationship. In some further embodiments, each monitoring device is arranged so that a central axis of a field of view of registration and/or detection of a monitoring device at least substantially aligns with a central axis of a respective enclosure of a received cell culture chamber device. In this way, one monitoring device is dedicated to register and/or detect an monitoring signal for one specific cell culture chamber device, which typically will enhance the quality of the respective monitoring signals and/or also enhance the registration and/or detection (e.g. viewing) of the specific culture chamber devices, and in particular of the content of any contained cell culture chamber devices. Additionally, it will also be assured (or at least greatly facilitated) that the monitoring device(s) and the cell culture chamber device(s) are correctly aligned (in X, Y, and Z dimensions), which is particularly significant given the typical short distances between them in such setups. Alternatively, one monitoring device is dedicated to register and/or detect an monitoring signal for a plurality of specific cell culture chamber devices thereby reducing the number of monitoring devices needed. This would e.g. be advantageous when the monitoring device is not inexpensive or when very high comparability is needed in the images collected from the different cell culture chamber devices. In such a case, the light respectively passing through said cell culture chamber devices may be guided using controllable lenses, prisms, mirrors, or other light guides to the monitoring device in such a way that the light entering the monitoring device has passed through or stems from only one cell culture chamber device at any one particular time. In other embodiments, the light could pass through more than one cell culture chamber device so that the light entering the monitoring device has passed through or stems from more than one cell culture chamber device at any one particular time. This would be the usual set-up for situations needing a reference or uses phase differences.

Additionally, the monitoring device(s) may readily enable detailed documentation of the contents of the cell culture chamber, for example in the form of single pictures or video sequences.

The illumination used could be of lower intensity and/or also lower wavelength, both of which may reduce any potential damage to the cells. Lighting could selectively be switched on when inspecting or documenting the content of the cell culture chamber device(s) using the monitoring device(s) and switched off after, which would reduce the period of time of light exposure. Additionally, a user may inspect, document, etc. the content of multiple cell culture chamber devices faster thereby also reducing the extent of light exposure.

Additionally, the monitoring device(s) may readily enable enhanced viewing (e.g. enlarged/zoomed, IR, transformed wavelength, etc. views) compared to manually looking through a glass panel or the like into the incubation chamber (even if lighting is present in the incubation chamber).

Alternatively, another (one or more) monitoring signal source(s) is/are used instead of light sources being configured to emit another type of monitoring signal, e.g. through a second end, or part or window thereof, into a respective enclosure, wherein the at least one monitoring device is configured to capture at least a part of the other type of monitoring signal transmitted through a first end, or part or window thereof, to outside the enclosure. This other type of monitoring device(s) may e.g. be configured for registration of sound or acoustic waves (e.g. ultrasound) or for registration of electromagnetic radiation of wavelengths different than those of visible light (e.g. x-rays, ultraviolet or infra-red).

In all or at least some of the embodiments for illumination of the cell culture chamber device and subsequent capture of the image of the device, the light may be led through one or more, e.g. controllable, features such as lenses, diffusers, filters, light guides, prisms, mirrors etc. as are commonly used for manipulating light. Some or all of these features may be independently exchangeable to enhance at least to some degree the image obtained and in so doing increase the ‘signal-to-noise’ ratio. Other features may be used for similar purposes when manipulating electromagnetic radiation of wavelengths different to visible light are also included herein.

In the illustrated embodiments, a light source is providing back-lighting while two (as an example; may be one or another number than two) light sources provide front-lighting of a cell culture chamber device and a cell culture chamber, which may enable simple viewing of the contents of the enclosure by a monitoring device.

The light source(s) may e.g. be LED light source(s) or any other suitable light source.

In some embodiments, where the drive unit comprises a plurality of light sources, they may be of the same type or alternatively of different types (e.g. emitting different wavelengths). In some further embodiments, the drive unit comprises a plurality of light sources being of at least two different types, where the at least two different types may be selected from the group of UV, visible, near IR, and IR light.

In some further embodiments, the light may be of restricted wavelengths and may be coherent (e.g. as in a laser) or alternatively not, in order to increase a signal to noise ratio.

In yet further embodiments, the illumination may be restricted in time, i.e. be pulsed, flashed, or be even a single pulse or flash so as to reduce the ‘background’ illumination and in doing so increasing the signal to noise ratio. Pulses may be of variable length of seconds or fractions of seconds, potentially down to the millisecond time interval for certain applications (e.g. for time resolved fluorescence). Certain light emitting compounds are unstable and break down upon illumination. Pulsed illumination as described would thus extend their usable lifetime and the length of the pulse could then be determined by the monitoring device to obtain suitable exposures.

In some embodiments, the incubator system comprises a light diffusor arranged in a light propagation path from the one or more light sources to or towards an enclosure of a received cell culture chamber device. In some further embodiments, the light diffusor is arranged in the propagation path adjacent to or at least near a received cell culture chamber device (e.g. adjacent to or near a second end of an enclosure). The light diffusor may e.g. be arranged in a cavity of the drive unit. The light diffusor will provide a more uniform lighting towards the enclosure and may therefore increase the quality of the backlighting and thereby the monitoring signal of the monitoring device(s).

In some embodiments, the incubator system comprises a light filter which, at least to some extent, permits light of particular wavelengths and blocks other wavelengths, arranged in a light propagation path from the one or more light sources to or towards an enclosure of a received cell culture chamber device. In some further embodiments, the light filter is arranged in the propagation path adjacent to or at least near a received cell culture chamber device. The light filter may e.g. be arranged in a cavity of the drive unit. The light filter may provide a more selective lighting from the enclosure and may therefore increase the ‘image signal to noise ratio’ and thereby enhance the monitoring signal of the monitoring device(s).

In some embodiments, the incubator system comprises a light filter which, at least to some extent, permits light of particular wavelengths and blocks other wavelengths, arranged in a light propagation path from the cell culture chamber device to or towards a monitoring device. In some further embodiments, the light filter is arranged in the propagation path adjacent to or at least near a monitoring device. The light filter may e.g. be arranged in a cavity of the drive unit or immediately in front of the monitoring device or receptor. The light filter may provide a more selective lighting from the enclosure and may therefore increase the image signal to noise ratio and thereby the monitoring signal of the monitoring device(s).

In some embodiments, a combination of diffusor and filter or filter sets is used at any position between the source of the illumination and its registration to obtain a high image signal to noise ratio thereby enhancing the monitoring signal of the monitoring device(s). Such diffusors and filters may e.g. be removable or exchangeable to match the requirements of the cells, sensor, or monitoring device.

The viewing and monitoring of the content of the enclosure(s) in this way significantly reduces the need for repeatedly opening and closing the incubation chamber disrupting the controlled environment therein (e.g. with respect to temperature, humidity, O₂, CO₂, simulated microgravity, shear stress, etc.). This in turn significantly reduces the perturbation of the cells inside any cell culture chamber devices in the incubation chamber.

The rotational drive unit(s) is respectively configured to rotate one (or more) received cell culture chamber device(s) about one, two, or three mutually substantially perpendicular axes. Incubators rotating about two or three such axes are sometimes also referred to as so-called random positioning machines. In some embodiments, the predetermined rotational axis is a predetermined central axis of the received cell culture chamber device. In some embodiments, the central axis of the received cell culture chamber device may be the same or coincide with the central axis of the enclosure of the received cell culture chamber device, i.e. if the enclosure is centrally arranged within the cell culture chamber device (which does not always need to be the case). Alternatively, the predetermined rotational axis is displaced from the central axis of the enclosure.

In some embodiments, the rotational drive unit(s) is/are clinostat drive unit(s).

In some embodiments, the incubation chamber is configured to contain the cell culture chamber devices in full (when received by the incubator). Alternatively, the incubation chamber is configured to contain only a part of the cell culture chamber devices, in particular at least a part, e.g. the whole, of the respective enclosure(s). In some embodiments, the cell culture chamber devices may e.g. be perfusion cell culture chamber devices (than can be self-sustained for prolonged periods of time e.g. up to about 14 days or more), comprising respective fresh and spent media reservoirs, drive element(s), etc. and it would be an advantage to have certain parts or components, such as the fresh and spent media reservoirs, the drive element(s), etc., outside the incubation chamber thereby reducing the risk of contamination and enabling easier cleaning. See e.g. Applicant's co-pending PCT patent application with application number PCT/EP2020/068632 for examples of perfusion cell culture chamber devices or bioreactors.

In some embodiments, the incubator(s) and/or the user interface device is further configured to perform data logging and/or documentation e.g. collecting and storing data such as temperature, humidity level, rotational speed, e.g. over time and e.g. including averages as well as duration and number of pauses (without rotation), etc. for at least some, e.g. all, of the cell culture chamber devices. This may e.g. be supplemented with video(s) and/or still image(s) and with data from image processing. The data of the data logging or documentation may e.g. be stored (e.g. also) in a cloud computing environment.

The monitoring signal may e.g. be a visual light signal and the monitoring device may e.g. be a monitoring device or the like configured to providing a video feed or video capture, (e.g. periodic) still images, etc. of any contained cell culture chamber devices. Alternatively, the monitoring signal is a different signal, e.g. as disclosed herein and the monitoring device would be suitable to collect this different signal. In addition, or alternatively, the above functionality is provided for other types of monitoring signal(s), e.g. as disclosed herein, than video and images.

According to the second aspect, one or more of these objectives is achieved, at least to an extent, by an incubator in the first aspect, further comprising
a unit, whether as hardware or software, designed to capture single images or single frames from a video, preferably at high resolution (a ‘frame-grabber’),
one or more processing units (which may or may not be an integral part of the incubator unit itself), one or more of which is enabled to analyse images in real time (or near real time),
a processor unit and one or more algorithms which can coordinate the activities of the image analysis being carried out
a logging system to collect the image-derived data for each cell culture chamber device an electronic memory and/or electronic storage, and
one or more signal transmitter and receiver communications elements configured to communicate with a network,
an element which can provide said data to the operator of the incubator.

The ID of a particular cell culture chamber device may be obtained automatically by capturing an image or video of one or more fiducial and/or identification markers or codes and performing appropriate image processing or other digital processing. In a similar manner, the presence of bubbles contained in a specific enclosure of a cell culture chamber device may also be obtained and presented by capturing an image or video. Therefore, in some embodiments, the enclosure and/or the cell culture chamber device further comprises one or more fiducial and/or identification markers, such as identification markings, barcodes, points of reference, etc. At least some of the fiducial and/or identification markers is/are preferably machine readable. This may e.g. be advantageously used in connection with monitoring using at least one monitoring device as disclosed herein, e.g. an imaging or vision system or device. The fiducial marker(s) enables determination of the orientation of the cell culture chamber device (and in particular of the enclosure) for use with at least one monitoring device as disclosed herein.

An identification marker is preferably unique to the particular cell culture chamber device that it is comprised by. In a further application, image analysis of cell culture chamber devices will read device-specific bar codes and ensure that the cell culture chamber device is rotated at the correct speed (irrespective of which ever axel in the incubator system the device is placed on). Image analysis of the bar code will also ensure that the data is correctly collected and filed for that device.

More specifically, in at least some embodiments, at least one or some of the cell culture chamber devices each further comprises one or more fiducial marks, bar codes, or similar marks, and wherein the one or more processing units is configured to recognise such marks using image analysis and to identify a respective cell culture chamber device in response thereto, and wherein the incubator system is configured to control a rotational position and/or a rotational speed of the respective cell culture chamber device, and/or to monitor the use of the respective cell culture chamber device.

In a particular embodiment, using device-specific bar codes, the incubator can alert the user that the cell culture chamber device itself is nearing the end of its effective time period. The alerts can be effected by a variety of means such as a pop-up window on the tablet, an e-mail or SMS to a computer or smart phone, or to multiple targets or persons (user, co-user, group leader, security personnel, etc.).

In another particular application of this embodiment, the image processing can provide an apparently stationary image of the rotating cell culture chamber, facilitating its inspection by the user. More specifically, in at least some embodiments, the one or more processing units is configured to maintain a video or one or more images of the video of a rotating cell culture chamber device in an apparently stationary position by counter-rotating by an amount determined in response to a rotation per minute or other rotational velocity value of the rotating cell culture chamber device.

The online feed video or the pictures may readily enable manual inspection of the state of contained spheroids, e.g. their size, their orbit, their rate of fall, etc., which might prompt a user to want to change, e.g. increase, the rotational speed, if for example the spheroids now have become larger and thereby heavier (prompting for an increased rotational speed).

In some further embodiments, the incubator(s) are further configured to carry out image analysis in real time or near real time in a non-invasive manner. In certain cases, where intensive processing capacity is needed, the image analysis tasks may be shared with external computers or supercomputers. The purpose of such image analysis is to provide the user with additional data on the culture in the cell culture chamber, to facilitate and accelerate data processing.

The user interface device may e.g. be configured for online monitoring of the first and/or any of the at least a second incubator. In some embodiments, the user interface device is configured to display in a user interface e.g. on a screen, a video online feed or a latest single or series of pictures for one or more of each incubator as obtained by the incubator via its respective monitoring device if comprising such as disclosed herein. Additional data, such as current rotation speed, rotational direction, ID, etc. for each particular cell culture chamber device may also be obtained and transmitted to the user interface device e.g. to be displayed on the device together with the video or image(s) for a respective cell culture chamber device. In addition to data that is specific to the cell culture chamber device(s), data for the respective incubators may also be obtained and provided for example one or more of: current temperature, current pH value, current humidity, current CO₂, O₂, and/or N₂ level(s), etc. The incubator system may e.g. also send an alert or alarm to the user interface device (or another connected external computational device) if certain one or more parameters is/are outside an acceptable range of values, above or below an accepted value, etc. (e.g. if the measured current temperature or CO₂ level exceeds a given temperature threshold or value, etc.).

Image analysis can also provide a non-invasive, real-time feedback to the user as to the pH of the culture, the amount of biomass (DNA protein, number of cells), the rate of growth, the size and standard deviation (reproducibility) of the spheroids or organoids in the cell culture chamber. In a particular embodiment, the incubator can alert the user that the culture is deviating from expected growth parameters. These alerts can be effected by a variety of means such as a pop-up window on the tablet, an e-mail or SMS to a computer or smart phone, or to multiple targets or persons (user, co-user, group leader, security personnel, etc.).

Image analysis can also be used to determine the area of the water beads in the image so that the user can be warned if the humidification system is running low on water.

In a further embodiment, the application of the light sources (both front- and back-lighting) and monitoring device(s) can be combined with the presence of sensors on the cell culture chamber, in the media, and on or in the cells or spheroids in the cell culture chamber to provide the user with a plethora of sensitive and precise data about the culture, or biological processes occurring therein in a non-invasive manner. Image analysis can provide the user with a real time feedback of the sensor output data (see e.g. FIG. 7D for an embodiment thereof).

Combinations of different aspects of the image analysis output data can be used to normalise the data collected. Combinations of the data from different cell culture devices (potentially in different incubators) can be used to compare control and treated samples. This processed data can also be provided to the user in real time.

According to further aspects and/or further embodiments is provided one or more processing units (e.g. of an incubator system and/or a computer as disclosed herein) configured to carry out one or more of the image analysis methods or steps thereof as disclosed herein. In some embodiments, the incubator(s) and/or the user interface device is further configured to perform data logging and/or documentation e.g. collecting and storing data such as rotational speed, biomass (DNA protein, number of cells), the rate of growth, the size and standard deviation (reproducibility), and any sensor output, each measurement being made over time and, for example, including averages as well as duration and number of pauses (without rotation), etc. for at least some, e.g. all, of the cell culture chamber devices. This may e.g. be supplemented with video(s) and/or still image(s) and with data from image analysis. The data of the data logging or documentation may e.g. be stored (e.g. also) in a cloud computing environment.

In this second aspect, the one or more processing units e.g. is/are configured to communicate via the network with at least one external computational device, e.g. one or more of a user interface device, a client and/or server computer or device, a network connected storage device, and/or one or more additional incubators.

In some embodiments, captured or obtained videos and/or pictures and/or other monitoring signals of the monitoring device(s) may be transmitted by the incubator to the external computational device, e.g. for presentation (e.g. remote viewing or remote online viewing), storage, and/or further digital processing. Such devices may store not only the raw data (and the time at which it was collected) but e.g. also the processed data and also the time at which it was utilised.

Suitable processing power and memory capacity can be found in current good quality tablets. Should higher processing speeds and/or memory capacities be needed these can be provided by computers or supercomputers.

In such embodiments, image analysis algorithms may be used to extract a variety of types of data from one or more images of the cell culture chamber device. In some cases, the image analysis may enable data collection that would either be taxing, arduous or even practically impossible for the operator or would have negatively impacted the growth of cells in the cell culture device.

In yet other cases the data may be used to automatically interpret or derive an estimate of the progress of growth and/or the effects of a treatment and in doing so enable the collection of unbiased data. Thus, the equipment may not only present raw read-out data but also provide processed data (e.g. including estimates of reliability or reproducibility of such data). Image analysis could e.g. be applied to the whole image or part(s) thereof, e.g. an area of the image comprising the cell culture chamber (i.e. where the spheroids are located). Reduction of the area to be analysed will correspondingly increase the image analysis speed and reduce power consumption. The following description of image analysis can be applied to the total image, or e.g. the three different colour components of the image (red, green and blue) used independently or in any weighted combinations. Use of one colour component or a derived monochrome or greyscale image will also increase the image analysis speed.

The images produced can be processed to provide detailed information about the cells in the cell culture chamber device without disturbing their growth (i.e. in a non-invasive manner). This allows such measurements to be made much more frequently than would be possible or permissible if the cell culture chamber device had to be removed from the incubator and inspected manually. More importantly, the image analysis may enable data collection which the cells could not tolerate if the cell culture chamber device needed to be removed from the incubator system in order for the measurement to be made. Such images could be processed on, for example, a second-to-second basis through short periods of time (minutes or hours) if relatively fast reactions were occurring in the cell culture chamber, such observations being practically impossible if the cell culture chamber had to be removed from the incubator system. In other cases, such images could be processed on a minute-by-minute basis through extended periods of time (days, weeks, or months) if so desired. Data from various cell culture chambers can be compared to follow the development of a culture or an experiment in real time or near-real time, further reducing the need for operator intervention and increasing the speed at which conclusions can be deduced.

Furthermore, the image analysis can generate a number of signals or data/information to the user which indicate the progress of the experiment, and most importantly indicate deviations from expectations.

There exists a wide array of different types of sensor which are all compatible with the growth of cells and can thus be included in the cell culture devices without significantly affecting the activity of such cells. These can be used to further collect additional data about the cell culture, its performance or response to provocation or treatment. The data acquired may require image analysis in a manner similar to visual images.

According to this second aspect, embodiments of the image analysis algorithm(s) start with processes like one or more of acquisition, enhancement, colour space conversion, and digital image transformation. Such images thus may e.g. need pre-treatment for morphological image analysis (smoothing, sharpening, focussing or adjustment of contrast and brightness and the removal of image noise (e.g. individual or small groups of pixels with a value significantly different to the (eight or more) pixels around them can be replaced with an average value of the eight or more pixels) followed by local image segmentation and object recognition. Many of the processes in image analysis can also be improved by employing machine learning (ML, that is computer programs that can access data and use it for their own learning, without being clearly programmed), deep learning (DL), or convolutional neural networking (CNN) but these processes need considerable processing power and are slower than traditional image analysis. However once learnt, these image processes can be in some cases faster or more efficient at performing particular tasks (e.g. dividing visual elements in the image (such as identifying or delineating an object from the ‘background’ or separating visually overlapping objects), sorting different elements into separate groups, etc.).

Image segmentation may be an early step in image analysis and may be used to divide an image into constituent parts (elements). These elements can include, but are not limited to, the cell culture chamber device, the cell culture chamber enclosure (e.g. in one particular embodiment as defined by a large, bright, essentially circular element of known diameter in the image; a bar code or similar (e.g. as defined by a series of light and dark bars, squares or rectangles in a rectangular area of known dimensions outside of, but at a known distance to and at a known angle to, the cell culture chamber) and a fiducial marker(s) of known position (inside or outside the cell culture chamber) and of known size and shape and a front plug of known size and shape of the cell culture chamber.

Once identified, the bar code can be read to identify unambiguously the precise cell culture chamber device. Should this cell culture chamber device be removed from one drive axel and returned to a different axel (potentially in a different incubator but typically in the same incubator cluster (i.e. controlled by the same processor tablet or computer), the software could then continue to process the cell culture chamber device with the same rotational speed as used on the previous axel and could continue to attribute data collected to the correct cell culture chamber device (and not a different or new one) and furthermore continue to analyse any ongoing or kinetic processes without dislocation of the data stream. This function is advantageous as it will suppress operator errors due to the repositioning of a cell culture device on a different axel in an incubator or incubator cluster.

Image processing can also for example stop any axel which does not hold a cell culture chamber device.

In additional specific embodiments, the incubator system may also keep track of how long a particular cell culture chamber device had been used (by logging the inception date and time and measuring against the elapsed time of use) and indicate to the user when the device needed to be exchanged e.g. because gas exchange membranes (for embodiments comprising such) or other were reaching the end of their functionality or usability This function is useful as it may ensure that the cell cultures are maintained in uniform conditions.

In some further embodiments, once the bar code or the fiducial marker was identified, the one or more processing units is configured to stop the cell culture chamber device in a particular orientation, for example with a circumferential port at the top. Once stopped, the spheroids in the cell culture chamber device would sediment to the bottom. The user could then e.g. move the cell culture chamber device to a sterile bench or other site without changing the orientation of the device so that for example the growth media could be changed more rapidly with minimal disturbance to the spheroids, or to facilitate the collection of spheroids.

According to a further embodiment, the image data is rotated so that when it is displayed it appears or is stationary. Thus the image data is processed in such a way that each image, or part of such (e.g. the part corresponding to the cell culture chamber device), was rotated backwards by an amount corresponding to the forward rotation occurring during the time between taking one image frame and the next. This could e.g. be performed by locating the bar code and/or the fiducial marker and keeping its/their position constant in the images to be presented. When the spheroids are in ‘stationary orbit’ relative to the cell culture chamber device, this would facilitate the observation of individual spheroids because they would appear to remain roughly motionless in the image, enabling a closer inspection. Maintaining in effect the spheroids essentially motionless would permit tracking of individual spheroids over extended periods of time and permit kinetic observation of biological processes in a single spheroid.

The rotation of images in general and as such is a well-known general image analysis process (being a special case of an affine transformation). The process may e.g. also perform translational transformations to better position the image of the rotating cell culture chamber (for example to centre the image of the cell culture device in the screen). Such image analysis would have to preferably occur within the time between one frame and the next in order to provide the smoothest video feed. Processing in which one or more than one frames are ‘skipped’ will result in a less smooth video but will give more time for the image analysis.

In some embodiments (according to the first and/or second and e.g. further aspects), the data extracted and/or derived from the one or more monitoring signals is or comprises one or more digital images and/or a digital video obtained of or for a contained cell culture chamber device, and wherein the incubator system is further configured to regulate the speed of rotation of the contained cell culture chamber device by performing image analysis on the one or more digital images and/or a digital video or parts thereof. In the following embodiments of changing the speed of rotation, the direction of rotation is clockwise. The functionality would need to be adjusted accordingly for anti-clockwise rotation (more or less, the regions mentioned in the following should or could be mirrored in relation to a generally vertical line of the relevant image). In some further embodiments, the image analysis is performed on an image comprising or being divided into a first region (see e.g. 32 in FIG. 5), a second region (see e.g. 33 in FIG. 5), and a third region (see e.g. 31 in FIG. 5), and wherein the incubator system is configured to rotate the contained cell culture chamber device about the respective predetermined axis in a clock-wise direction and to
decrease the speed of rotation of the contained cell culture chamber device if a sum of pixel intensity values per unit area, or other image intensity metric or similar, for the first region, alone or together with an added predetermined positive tolerance value (x %), is greater than a sum of pixel intensity values per unit area, or other image intensity metric, for the second region;
increase the speed of rotation of the contained cell culture chamber device if a sum of pixel intensity values per unit area, or other image intensity metric or similar, for the third region, alone or together with an added predetermined positive tolerance value, is greater than a sum of pixel intensity values per unit area, or other image intensity metric, for the first region; and/or
maintain the speed of rotation of the contained cell culture chamber device for any other case.

In some embodiments, the second region (see e.g. 33 in FIG. 5) is a substantially circular and central region encompassing a centre of the cell culture chamber, the first region (see e.g. 32 in FIG. 5) is a substantially outer ring-like part being adjacent to and partly encompassing the second region (‘up’ and to the ‘sides’), and the third region (see e.g. 31 in FIG. 5) is a substantially outer ring-like part being adjacent to and partly encompassing the second region (‘below’ or ‘under’). In at least some embodiments, the first and third region will fully circumscribe and enclose the second region and the first region has a larger extent or area than the third region.

Alternatively, the image analysis is performed on an image comprising or being divided into a fourth region (see e.g. 37 in FIG. 6), a fifth region (see e.g. 36 in FIG. 6), a sixth region (see e.g. 34 in FIG. 6), and a seventh region (see e.g. 35 in FIG. 6), and wherein the incubator system is configured to rotate the contained cell culture chamber device about the respective predetermined axis in a clock-wise direction and to
decrease the speed of rotation of the contained cell culture chamber device if a sum of pixel intensity values per unit area, or other image intensity metric or similar, for the fourth region added together with a sum of pixel intensity values per unit area, or other image intensity metric or similar, for the fifth region, by themselves or added together with a predetermined positive tolerance value, is less than a sum of pixel intensity values per unit area, or other image intensity metric or similar, for the sixth region added together with a sum of pixel intensity values per unit area, or other image intensity metric or similar, for the seventh region,
increase the speed of rotation of the contained cell culture chamber device if a sum of pixel intensity values per unit area, or other image intensity metric or similar, for the fifth region added together with a sum of pixel intensity values per unit area, or other image intensity metric or similar, for the fourth region, is greater than a sum of pixel intensity values per unit area, or other image intensity metric or similar, for the sixth region added together with a sum of pixel intensity values per unit area, or other image intensity metric or similar, for the seventh region by themselves or added together with a predetermined positive tolerance value, and/or
maintain the speed of rotation of the contained cell culture chamber device for any other case.

In some embodiments, the fourth region (see e.g. 37 in FIG. 6), the fifth region (see e.g. 36 in FIG. 6), the sixth region (see e.g. 34 in FIG. 6), and the seventh region (see e.g. 35 in FIG. 6) are of equal size and defined by two lines crossing each other in the middle of the cell culture chamber (image) at an angle about 90° and the cell culture chamber image at an angle of greater than 45° (line a - - a) where the fourth region (in the image) would be mostly ‘up and left’, the fifth region would be mostly ‘down and left’, the sixth region would be mostly ‘up and right’, and the seventh would be mostly ‘down and right’. The two crossing lines would be rotated a certain predetermined amount in relation to horizontal and vertical depending on the rotation direction (clock-wise or anti-clock-wise). For clock-wise rotation, the two crossing lines would be rotated a small amount anti-clock-wise (see e.g. FIG. 6).

In additional embodiments, the determination(s) of whether the speed of rotation is to be increased or decreased is made by comparing derived data for ‘only’ the fifth region vs. the sixth region rather than comparing data derived for the fourth and fifth regions vs. the data derived for the sixth seventh region. This is for example suitable (while simpler) for actual speeds being a ‘long way’ from a correct speed. For actual speeds ‘closer’ to a correct speed, comparing data derived for the fourth and fifth regions vs. the data derived for the sixth seventh region is preferred or even required.

In alternative embodiments of adjusting the speed of rotation mentioned above (and elsewhere), an average pixel intensity value for a particular region is derived and used rather than deriving and using a sum of pixel intensity values per unit area for a particular region. In further alternative embodiments, other ways of determining or estimating the amount or mass of cell clusters, spheroids, etc. contained in the various regions of the cell culture chamber may be used. Regions herein are typically equally referred to as zones.

It is noted, that the two above alternative embodiments (and further variations/embodiments thereof) of changing or adjusting the speed of rotation, assumes—or at least works best with—substantial uniform lighting of what the image (ultimately having or providing the regions) is obtained of. If the lighting is not sufficiently uniform, this needs to be accommodated for in a suitable way as generally known, e.g. by subtracting a (local) background pixel intensity value from the pixels of the regions before comparison/analysis. Furthermore, the comparisons (‘greater than’ and ‘less than’) of these embodiments given above also assumes or works best with front lighting (where spheroids, cell clusters, etc. appear lighter than their surroundings). If back lighting is used (where spheroids, cell clusters, etc. appear darker than their surroundings), ‘larger than’ should be replaced with ‘less than’ and ‘less than’ should be replaced with ‘larger than’ in the above comparisons. Or the image or regions could simply be digitally converted to a negative version of itself (where light areas becomes dark and vice versa) prior to executing the comparisons. Furthermore, if the regions are not generally of the same size, there is no need to sum pixel intensity values (or any other used function or metric) per unit area; rather (in the case of the regions being of a substantially similar size), the pixel intensity values of the respective regions can then simply be summed and immediately compared.

For embodiments with back lighting instead of front lighting, the comparisons could be according to the following.

In some such (back light embodiments), the image analysis is performed on an image comprising or being divided into a first region (see e.g. 32 in FIG. 5), a second region (see e.g. 33 in FIG. 5), and a third region (see e.g. 31 in FIG. 5), and wherein the incubator system is configured to rotate the contained cell culture chamber device about the respective predetermined axis in a clock-wise direction and to
decrease the speed of rotation of the contained cell culture chamber device if a sum of pixel intensity values per unit area, or other image intensity metric or similar, for the first region, alone or together with an added predetermined positive tolerance value (x %), is less than a sum of pixel intensity values per unit area, or other image intensity metric, for the second region;
increase the speed of rotation of the contained cell culture chamber device if a sum of pixel intensity values per unit area, or other image intensity metric or similar, for the third region, alone or together with an added predetermined positive tolerance value, is less than a sum of pixel intensity values per unit area, or other image intensity metric, for the first region; and/or
maintain the speed of rotation of the contained cell culture chamber device for any other case.

And in some such alternative (back lighting) embodiments, the image analysis is performed on an image comprising or being divided into a fourth region (see e.g. 37 in FIG. 6), a fifth region (see e.g. 36 in FIG. 6), a sixth region (see e.g. 34 in FIG. 6), and a seventh region (see e.g. 35 in FIG. 6), and wherein the incubator system is configured to rotate the contained cell culture chamber device about the respective predetermined axis in a clock-wise direction and to
decrease the speed of rotation of the contained cell culture chamber device if a sum of pixel intensity values per unit area, or other image intensity metric or similar, for the fourth region added together with a sum of pixel intensity values per unit area, or other image intensity metric or similar, for the fifth region, by themselves or added together with a predetermined positive tolerance value, is larger than a sum of pixel intensity values per unit area, or other image intensity metric or similar, for the sixth region added together with a sum of pixel intensity values per unit area, or other image intensity metric or similar, for the seventh region,
increase the speed of rotation of the contained cell culture chamber device if a sum of pixel intensity values per unit area, or other image intensity metric or similar, for the fifth region added together with a sum of pixel intensity values per unit area, or other image intensity metric or similar, for the fourth region, is less than a sum of pixel intensity values per unit area, or other image intensity metric or similar, for the sixth region added together with a sum of pixel intensity values per unit area, or other image intensity metric or similar, for the seventh region by themselves or added together with a predetermined positive tolerance value, and/or
maintain the speed of rotation of the contained cell culture chamber device for any other case.

In some embodiments (according to the first and/or second and e.g. further aspects), the incubator system comprises at least two axels or drive units, each configured to rotate a respectively connected or received cell culture chamber device step, wherein the incubator system is further configured to identify a particular cell culture chamber device (e.g. using image analysis and fiducial or other markers as disclosed herein) connected or received on a particular axel or by a particular drive unit and adjust the rotational speed of the particular axel or the particular drive unit to a rotational speed associated with the connected or received particular cell culture chamber device.

In yet another embodiment of image analysis, an additional operation would be to identify elements within images of the cell culture chamber enclosure and such restriction of the processing area to a part of the image would increase processing speed. This could be done using one or more of a variety of image analysis approaches collectively known as segmentation (using either universal or local background subtraction (where the background can be defined by for example ‘rolling ball techniques’, local minima or maxima or adaptive Gaussian thresholding). There are a wide variety of segmentation approaches known in the field of image analysis, including edge-based segmentation (‘edge-detecting’ ‘corner detecting’ ‘blob-detecting’ algorithms), morphological segmentation, graphic-based segmentation filters, cluster-based segmentation and probabilistic segmentation. Algorithms may contain ‘tolerance’, that is to say that if ‘edges’ are not detected all the way around an element, then the program can interpolate to complete the element edge.

Once elements are identified in the images, an additional set of algorithms may be applied to sharpen or smooth the element, to determine its area and volume (as defined as the integrated optical density (IOD—the sum of the pixels within the element minus the sum of the (local) background around the element), minimum and maximum axes (not necessarily in the Cartesian X, Y and Z directions), average diameter, smoothness, circumference, circularity and actual position in the cell culture chamber area (Cartesian X, Y and potentially Z values (the latter may e.g. be determined by focussing algorithms or similar)) with respect to for example to a point on one of the fiducial markers or the front port or other suitable point like a corner of the image.

Elements with, for example, low circularity, large circumference-to-area ratios or large differences in the minimum and maximum axes can be further analysed to for example, attempt to resolve the image of two (or more) overlapping spheriods. This can be done by for example pinching, division along the minimum axis, saddle-division, or water-shedding techniques or any of the above described segmentation approaches. These techniques may e.g. be repeated or used in different combinations in order to resolve complex elements.

According to a further development in this embodiment, once elements have been resolved, these elements can then be grouped and sub-grouped e.g. according to any combination of the above-mentioned parameters. Elements below certain threshold values can be excluded (e.g. image noise). Elements that have a large size to volume (IOD) ratio and preferentially located at the top of an image, may represent air-bubbles, or at the bottom may represent cellular debris, that can also be excluded from further analysis. Remaining elements can then be sorted into subgroups for example based on their average diameter. In many instances, a preferable spheroid culture will be one where the spheroids are uniform in size and shape because it would be expected that these will respond more uniformly to treatment or other processing. In other words, the spheroid culture will contain elements (classified as spheroids) with similar X, Y and Z dimensions, high circularity (spheroidicity), a particular average diameter (which will depend on the age of the culture or the number of cells used to initiate the spheroids) with a relatively small standard deviation around this value. Conversely, a poor culture of spheroids will have element-based groups with larger standard deviation of X, Y and Z dimensions, lower circularity (spheroidicity) and diameters around the group average X-Y-Z, circularity (spheroidicity) and diameter. It is possible that there are more than one group, such additional groups might represent groups of spheroids that could not be resolved by the image analysis algorithms or might actually be clumps of spheroids in the cell culture chamber or two different populations of spheroids.

Image analysis of successive images can be used to derive trajectory data about individual elements (e.g. spheroids). This information could be used, for example, in an automatic feedback loop to regulate the speed of rotation of the cell culture chamber to match user requirements.

Useful data and information may e.g. be extracted in an area of the image inside of the cell culture chamber but outside of all of the elements in the image (and also outside of possible edge effects around elements and the edge of the cell culture chamber and any associated ports). For example, compounds like phenol red, bromo-cresol purple or fuchsin can be added to cell culture media to indicate the pH of the media. The average colour of the area of the image outside of all elements can thus be used to estimate the pH of the medium (see e.g. https://www.biotek.com/resources/application-notes/using-phenol-red-to-assess-ph-in-tissue-culture-media/). This may, for example, use a look-up table (LUT) specific for the coloured compound and the specific growth media used, such LUT being either provided with the incubator system, derived by measurements of samples provided by the user or simply entered by the user.

Taking phenol red as an example, cell produce waste products which will slowly decrease the pH, gradually turning the solution from red (at pH 8.0) to orange (at pH 7.0) and then yellow (at pH 6.0) (see https://www.testallwater.co.uk/blog/post/what-is-phenol-red-in-swimming-pools/).

The media colour can, for example, be monitored over time by image analysis and a change to a defined value is an indication that the media needs replacing. The program can be configured to then alert the user of this fact.

Waste products produced by dying cells or overgrowth of contaminating microorganisms will cause a more rapid decrease in pH, also leading to the same change in the indicator colour. In the event that the colour change is more rapid than routinely seen, the program can be configured to notify the user that there may be a microbial contamination or (unexpected) cell death occurring in the cell culture chamber.

Image analysis can also provide a non-invasive, real-time feedback to the user as to the pH of the culture, the amount of biomass (DNA protein, number of cells), the rate of growth, the size and standard deviation (reproducibility) of the spheroids or organoids in the cell culture chamber. In a particular embodiment, the incubator can alert the user that the culture is deviating from expected growth parameters. These alerts can be effected by a variety of means such as a pop-up window on the tablet, an e-mail or SMS to a computer or smart phone, or to multiple targets or persons (user, co-user, group leader, security personnel, etc.).

In a third aspect, one or more of the objectives stated above is achieved, at least to an extent, if the incubator system described above in the second aspect, together with the image analysis functionality further comprises:
software implementing image analysis which can extract data from the images of the cell culture chamber devices,
an element or device enabling using some of the image-derived data to modify the operation of the incubator via software-regulated electromechanical components for the regulation of, for example: media exchange for said device; or the speed of rotation of the cell culture chamber device; or the illumination of said device for the collection of data (including sensor readout) optionally at prespecified times,
a controller which can coordinate the various processor activities,
a logging system to collect and record at least some the modifications made to the operation of the incubator
a means to provide said data to the operator of the incubator.

In this third aspect, the incubator system, its imaging and image analysis capacity can be used to regulate the operation of the incubator system itself in order to optimise or otherwise adjust culture conditions. This will at least to some extent accelerate and facilitate the work of the user, minimise user interaction and in doing so increase the reproducibility of the analysis carried out. Thus, without user intervention, analysis of images collected can modify the performance of the incubator in some way as to improve or adapt the culture conditions for said culture.

In one particular embodiment, and using the example given above where image analysis is used to measure the colour of the media, then if the incubator system is of the ‘perfusion’ type, i.e. is capable of automatically exchanging at least part of the media, the colour change detected in the media can be used to activate a mechanism which causes an exchange of at least part of the media. This would result in the media being diluted with fresh media and the colour of the media in the cell culture device thus changing back towards a desired colour. Once the desired colour is reached, the mechanism exchanging the media would stop. Such regulation need not be regulated by the colour of the media but could be regulated for example by any other sensors present.

In another particular embodiment, image analysis can automatically and without interference by the user, adjust the regulation of the speed of rotation of the cell culture chamber device so that the spheroids are grown in ‘stationary orbit’ conditions, such adjustments would be difficult to achieve by the user because the adjustments may need to be carried out numerous times each 24 hours for days, weeks or months. There are several different algorithms which can be used to achieve this. The decision as to which algorithm to use can be made on the fly by the incubator processor itself in response to image analysis of the cells or spheroids in the cell culture chamber in question.

This improvement in the regulation of the speed of rotation of the cell culture chamber device will lead to a further increase in the reproducibility in and between experiments and to a reduction in the subjective variation which advertently or inadvertently is introduced by a user.

According to this embodiment, the distribution, obtained by image analysis, of the elements grouped as spheroids can be used to regulate the speed of rotation of the clinostat which is turning the cell culture chamber device. As the spheroids are cultured, they get larger due to the proliferation of cells. As they get larger, they also get heavier relative to the volume of water they displace. If the speed of rotation of the cell culture device is left unchanged, the spheroids will experience increased shear and collision forces, and decreased gas and nutrient exchange, all of which may be detrimental to their growth.

In practical terms, to prevent the growing spheroids sinking to the bottom as they get bigger, it is necessary to increase the rpm of the clinostat to achieve the ‘stationary orbit’ referred to above. For example, a ‘young culture’ of dispersed single cells needs to be rotated by about 4 rpm. With time, these cells will form spheroids. After about 21 days the cell culture chamber device needs to be turned at about 18 rpm while after about 42 days the speed needs to be about 40 rpm (actual speeds will depend on the cell type and the media used). One of the ‘outputs’ of image analysis can be used to adjust the rpm of the motor driving the cell culture chamber device in the image until the spheroids are in stationary orbit. Such calculations should be repeated frequently (for example initially every 10-30 minutes) until the spheroids achieve their stationary orbit. Thereafter the calculations should be repeated less and less frequently. Algorithms could also allow the frequency of repeat to increase (if for example the degree of change of the rpm needed increased following treatment of the spheroids). Each adjustment of the rpm may be logged.

Finding the rpm at which spheroids reach their stationary orbit can be achieved in a number of approaches and the following describes four examples. In these examples it is assumed that the cell culture device is rotating in the clockwise direction, that the top of the image corresponds to the physical top of the cell culture chamber, there are no air bubbles present and that spheroids appear darker than the background (i.e. they are subjected to back-illumination). Common to the examples provided below, is that the calculations are used to control the speed of rotation (rpm) of the motor driving the cell culture chamber device of the image. Examples of the distribution of spheroids (of the same age and in the same media) in a cell culture chamber device run at different speeds are provided in FIG. 4.

In the first example, the cell culture chamber image is e.g. divided up into zones, parts, regions, segments, etc. shown in FIG. 5 (said zones need not be of the same area and can be overlapping). The actual number, shape and size of the zones in the figure are only exemplary and in practice can be different to that shown. Furthermore, the positions of these zones will depend on the direction of rotation (clockwise or anticlockwise). All pixel position values lying in zone 30 (a circular zone very close to the edges of the cell culture chamber image) may be excluded from further calculation due to potential edge effects (for example optical aberrations and reflections at the edge of the chamber). Other elements (for example a front port (24)) can be excluded in the same way. The average pixel intensity (API) is then calculated for zone 31 and the remaining part of the cell culture chamber (33+32). If the API for zone 31 is smaller than the API for zone 33+32 then this indicates that the spheroids are predominantly found in the bottom of the cell culture chamber indicating that the rpm is too low (the situation illustrated in FIG. 3 as ‘very slow’) (in this example spheroids absorb light and are therefore dark and thus contribute low ‘pixel values’ to the high (bright) background values). This would lead to the rpm of the motor being increased. The actual position of zone 31 will depend on the actual rpm and the viscosity of the medium used. At very low rpm speeds, zone 31 will be found at the bottom of the cell culture chamber image, while at increasingly higher speeds, zone 31 will be increasingly displaced, in the direction of rotation from the bottom.

In an algorithm complementary to the above, zone 32 represents an annular zone of the outer region of the cell culture chamber image (and in this case includes zone 31). If the API of zone 32+31 is smaller than the remaining zone (33) then the spheroids are located towards the outer edges of the cell culture chamber indicating that the spheroids are being pressed out by centripetal acceleration, (as illustrated in FIG. 3 by ‘very fast’) i.e. that the rpm is set too high and needs to be decreased.

Thus a combination of these two image analysis subroutines, one which increases the speed in situations where the speed is to low and the other which decreases the speed when the speed is too high, would lead to an improved regulation of the speed of rpm in which the spheroids would be maintained distributed more or less uniformly within the cell culture chamber (as illustrated in FIG. 3 by ‘correct’.

Other zone combinations could be used for a similar ‘feed-back regulation’. These could be that zone 31 could be the lower semicircle (zones 36+37 in FIG. 6) and 32+33 could be the upper semicircle (zones 34+35 in FIG. 6—the actual position of the semicircle needs to be displaced in the direction of rotation so that the dividing line would not be horizontal but inclined by some degree (such degree will depend on the actual rpm and the viscosity of the medium used)). In this situation, an API of the lower semicircle (zones 36+37) smaller that the API of the upper semicircle (zones 34+55) would indicate that the rpm is too low.

Another option would be that the cell culture chamber image be divided into four essentially equal zones (FIG. 6, zones 34, 35, 36 and 37) divided by two lines crossing each other in the middle of the cell culture chamber image at an angle about 90° and the cell culture chamber image at an angle of greater than 45° (line a - - a) to the vertical. If the API of zone 36 is greater than 34 or the API of 37 is greater than zone 35 then the rpm should be increased. If air bubbles are present, then the zones can be delimited to exclude the region in which the air bubbles would be found (at the top of the cell culture chamber mainly in zone 34).

Other methods of calculation can reach the same result but the above method has the advantage that by calculating the API for zones, it is not necessary to adjust for differences in area (for example due to the presence of the front port image element (24)), nor it is necessary to ‘identify’ spheroids as specific image elements.

In the second example, using the image analysis algorithms described above to identify the spheroids, the number, area or volume of spheroids found per unit area within the zones can be used instead of the API to achieve the same results.

In a third example, the four virtual zones (shown in FIG. 5) could be rotated at the same speed as the cell culture chamber device. In this case, the correct rpm speed would be when either the API or the number (or other attribute) of the spheroids in each of the four zones remained the least variable. At the ideal speed, the spheroids do not move, or move relatively slowly, relative to the cell culture chamber device and thus the API (or other attribute selected) for the various zones would remain unchanging. Slower speeds results in the spheroids tumbling in the cell culture chamber device (shown in FIG. 3 as ‘too slow’). Thus, the rpm speed could be reduced until the tumbling motion was just detected, and then the rpm speed increased back to the previous value.

In a fourth example, which can be used for fine adjustment of rpm, by combining the maintenance of image position (as described above where the image of the cell culture chamber is rotated backwards at the same speed as the cell culture chamber is rotating forwards) together with tracking of the individual spheroids, the algorithm could measure the average vector distance (irrespective of the angle) travelled by all spheroids and increase the speed of rpm. If this led to a decrease in this average vector distance, the algorithm could make a further increase in the speed. If, however this led to an increase in the average vector distance, the algorithm should decrease the speed of rpm.

Such algorithms could use the average of one or more than one image each time. It could run periodically, the periodicity could be determined by the degree of change made during the previous run of the algorithm.

Approaches described similar to the first example would have the advantage that much less computing would need to be carried out: i.e. this could be calculated more rapidly. This approach could be used for cultures where the spheroids are of high quality and uniformity (as defined using some of the parameters mentioned above e.g. standard deviation (SD) of the average diameter, SD of their X, Y or the difference between X and Y dimensions, or SD of circularity).

Approaches described similar to the second, third and fourth examples would have advantages when the quality and uniformity are not so high. Using these approaches, elements that are not spheroids are excluded from the calculations, resulting in an improved rpm adjustment for the spheroids.

Approaches described similar to the third or fourth would have the advantage that they provide a fine adjustment of the rpm. However, they require significant computing power and therefore may be utilised only when other approaches prove not precise enough (as defined in this case as providing a significantly oscillating rpm over time). For random positioning devices, i.e. clinostats where the cell culture chamber device is rotated in two or three mutually perpendicular directions, the Z dimension would need to be taken into these calculations in order to adjust the rpm.

In other words, there are a number of approaches in which image analysis derived measurements can be used to define which sort of subroutine is used to adjust the rpm of the cell culture chamber device.

In a further embodiment, the application of the light sources (both front- and back-lighting) and monitoring device(s) can be combined with the presence of sensors on the cell culture chamber, in the media, and on or in the cells or spheroids in the cell culture chamber to provide the user with a plethora of sensitive and precise data about the culture, or biological processes occurring therein. Image analysis can provide the user with a real time feedback of the sensor output data.

Thus, in another application of this embodiment, the area of the elements grouped as spheroids by the image analysis, can be used to provide an estimate of the amount of DNA, RNA, protein or cell numbers present in a spheroid or in the cell culture chamber device as a whole without removing the cell culture chamber device from the incubator system and also without stopping its rotation. Both serve to preserve the growth conditions for the spheroids and thus to minimise perturbations of the cells.

In their publications, Fey and Wrzesinski, Wrzesinski et al., and Fey et al., removed the bioreactor from the incubator and placed it, stationary, under a camera system (thus negatively affecting the growth of the cells). They then took pictures of the cultures and used a look up table (LUT) in 2012 (PMID 22454432), in 2014 (PMID 25222612) and again in 2020 (PMID 32905230—see supplemental information table 1 in that publication) which they have used to convert the average ‘shadow area’ of a spheroid (as they refer to it in those publications, but here just defined as the area of the element) to the number of cells present, amount of DNA or protein in the spheroid. The precise values in the LUT will depend on that actual cell line and the growth conditions being used, but the principle is the same for any cell type or growth condition. Knowing the area of each element grouped as ‘a spheroid’, it is thus possible to deduce the total number of cells, or amount of DNA or protein in a cell culture chamber device non-invasively. In the aspect described here, these measurements can be carried out with minimal perturbation of the cells allowing the measurements to be made frequently if so desired.

If this measurement is repeated at intervals, it is possible to follow the proliferation of the cells in the cell culture chamber over time and thus present the data to the user with minimal user interaction or effort.

Furthermore, the growth curve can then be automatically compared to previous growth curves and if the curve deviates from the ‘normal’ growth curve by a predetermined degree, the program can issue a notification message to the user.

Such information could also be used in a perfusion type of instrument to for example increase the rate of medium exchange so that the growing population does not become deficient for specific nutrients.

If the spheroids in the cell culture chamber have been treated in any way (for example with a compound like a drug, candidate drug, or a ‘hit’ or a mixture of compounds like a plant extract, a change in the growth media, or by changing the temperature, pH or irradiating the cell culture chamber with electromagnetic radiation (of a wavelength(s) to which it is at least partially transparent) it is possible to non-invasively measure the effect of the treatment on the proliferation of the spheroids. This ‘treated’ growth curve can then be compared to (previous) control ‘untreated’ growth curves and if the curve deviates from the control growth curve, the program can issue a notification message to the user. Furthermore, the effect of including or removing particular cell types to the cell culture chamber can be followed in a similar manner.

According to another embodiment, many types of sensors can be used in conjunction with the cell culture chambers described here in order to probe specific questions. Sensors have not been built into (or used in conjunction with) clinostat-type cell culture devices, often because there has been an inadequate or inhomogeneous light or electromagnetic radiation path into, through, or out of the cell culture chamber. Inclusion of such sensors, electromagnetic radiation (or other) sources and monitoring devices provides the significant advantage of being able to collect biological data from a cell culture without disturbing the growth environment provided to the cells allowing such measurements to be made frequently, repetitively and automatically. The path of the electromagnetic radiation, at least between the source of the electromagnetic radiation and the monitoring device should be substantially optically unobstructed and uniform (except for devices like diffusers which may be included to enhance the uniformity of illumination, and the sensor itself).

Ideally, these sensors should be non-toxic (as defined by the concentration present, not affecting the growth of the cells or chemically reacting with substances being investigated in the cell culture device). It is also possible to exchange them either for a similar sensor or for a sensor measuring a different compound or attribute. Sensors need not give a graduated readout but could be constructed so as to give a particular signal when a certain condition is reached. This could be rotational or oxidative stress, pH or any other attribute. The output of such sensors is often clearest (have the highest signal to noise ratio) when the sensor is illuminated with light or other electromagnetic radiation of a particular wavelength, or the sensor produces (emits or re-radiates at a different wavelength) light or other electromagnetic radiation of a particular wavelength.

In some embodiments, in particular those in which sensors are employed, the LED or laser light source could be selected to provide light of a particular wavelength, or the LED or laser employed could be tuneable to produce light of different wavelengths. Tuneable lasers are commercially available from numerous sources. The use of additional features (e.g. filters, diffraction gratings) to permit or block specific wavelengths is an optional approach to enhance the signal-to-noise ratio.

Some of the sensors employ a compound (for example which is sensitive to the pH, partial pressure of oxygen, or glucose) which is either applied to the inside surface of a cell culture chamber as a ‘spot’ or as part of a small sticker or pad, (such inside surface can also with advantage include the surface of a plug inserted into the port and in contact with the culture media). Illumination of this sensor results in the generation of a signal (usually electromagnetic) which can be captured by a suitable receptor (for example a monitoring device or radio receiver). Such an example of this is the ‘contactless’ oxygen sensor equipment called Fibox 4 trace (meter), optical fibre and Oxygen Sensor Spot SP-PSt6-NAU and produced by PreSens (Germany). PreSence also produce similar sensors for pH and CO₂.

Numerous glucose sensors are available which can be used to measure the concentration of glucose in the cell culture chamber.

Cells in the spheroids can use glucose as one of their energy sources (glucose is typically the primary energy source) and thus with time the activity of cells will cause the level of glucose in the media to fall. If the incubator system is of the ‘perfusion’ type, i.e. is capable of automatically exchanging at least part of the media, data relating to the level of glucose can be used to activate the mechanism which exchanges at least part of the media and by doing so this will result in an increase in the glucose levels.

In other embodiments, other types of sensors can be compounds that are added to the growth media and can be read in a similar manner (examples are the phenol red, bromo-cresol purple and fuchsin mentioned above).

In yet other embodiments, other types of sensors can be substances which are attached to (or adsorbed to) or absorbed into the cell (outside or inside), or which are soluble in the cell or parts of the cell (e.g. soluble in the membranes or particular membranes). Yet other sensors are part of the cell (incorporated into a lipid, protein, nucleic acid or any other component found in the cell) and can be read in a similar manner to that described above. There are numerous examples of this including (but not limited to) fluorescent dyes (e.g. fluorescent proteins (e.g. Scott, 2018 doi: 10.1038/s41598-017-18045-y or Bukhari 2019, doi: 10.1016/j.tcb.2019.08.004), labelled antibodies (available from many companies for example: Creative Biolabs, Nanostring, Antibodies Online and Genescript (the latter offer a service fluorescently labelling antibodies); binary dyes (e.g. Marti et al., 2007 doi:10.1016/j.tet.2006.08.109), chemical or fluorescent nanosensors (e.g. Ahmad et al., 2020 doi.org/10.1038/s41598-020-57654-y and Fu and Ma 2020 doi.org/10.1039/DONR02844D) or aptamers, or other compounds. These sensors can be read in a wide variety of ways. These include chemiluminescence, fluorescence, Fluorescence Resonance Energy Transfer (FRET e.g. Berchner-Pfannschmidt et al., 2008 doi: 10.1183/09031936.00013408), Bioluminescence Resonance Energy Transfer (BRET), or by using Organic Thin-Film Transistors (OTFTs) (including Organic Field-Effect Transistors (OFETs) and Organic ElectroChemical Transistors (OECTs)). Some of the nanoparticle biochemical sensors for compounds like glucose, glutathione, choline, NAD⁺ lactate, triglycerides, urea, proteins like IgG and C-reactive protein and steroids are reviewed by EI-Ansary and Faddah in 2010 (doi: 10.2147/NSA.S8199).

Of all of the nanosensors, nucleotide (DNA, RNA, LNA etc.) aptamers are unique in that a particular nucleotide sequence can be selected which binds selectively with a particular molecule (e.g. antibiotics, CEA or ATP) or part of a molecule (e.g. an epitope or a posttranslational modification). The nanosensor sequence can then be ‘tuned’ (by small modifications to the sequence) to a particular concentration range of that molecule and provide real-time feedback. Some sensors are ‘dual’ in that within the same construct, they emit two ‘reporter’ fluorescence wavelengths, one that is constitutively active and the other that is facultatively active (such activity being dependent on the concentration of the particular molecule being studied). Such dual systems have the advantage that it is possible to correct for the actual amount of sensor present in the cell and can thus provide a means to normalise readout between different cultures irrespective of how much of the sensor is incorporated.

Thus, in yet another embodiment, sensors can be used to provide data on physical, chemical and biological processes occurring in the cell or as a result of the cells activity. Depending on the nature of the sensor, it can be ‘interrogated’ at operator ‘request’ or at pre-programmed time intervals (such intervals may be as frequent as the sensor allows) throughout seconds, minutes, hours, days or longer allowing the operator to follow kinetic processes within the cell culture chamber in a non-invasive manner. The data derived from the sensor output can in some cases (as described above) be used to regulate the growth environment of the spheroids, whether this is to adjust the supply of fresh media, the rate of rotation of the cell culture chamber device, the frequency of sensor interrogation or some other parameter.

Since there are many types of sensor and they can function independently of each other and may absorb and emit light of different wavelengths, it is quite possible to have combinations of more than one sensor present in a particular cell culture at any one time. For example, if two different colour sensors were built into two different types of cell, it would be possible to monitor the proportions of the cell types in the culture. If two different colour sensors are built into the same cell they can be used to follow the activities of two different biochemical pathways (e.g. Bervoets Charlier, 2019 https://doi.org/10.1093/femsre/fuz001 and Rosenthal et al., 2018, DOI: https://doi.org/10.7554/eLife.33099).

Combinations of different aspects of the image analysis output can be used to normalise the data collected. Combinations of the data from different cell culture devices (potentially one or more than one device in each group, and potentially in different incubators) can be used to compare control and treated samples. This processed data can also be provided to the user in real time.

Thus, according to a further embodiment, parts of the data can be normalised by using the amount of DNA, protein or cell number (or any other sensor parameter), calculated as described above using for example a LUT and the shadow area of the spheroids. Normalisation of the data in this way allows the effect of for example cell proliferation over time to be excluded from the measured sensor output. The measurements for normalisation can be carried out at the same time (or shortly before or after) sensor interrogation to produce the most accurate data. However, if for example the cell proliferation is slow (relative to the process being investigated), and the sensor interrogation is frequent or carried out over a short time period, it may be acceptable to use one set of normalisation data for all sensor data.

In a particular application of this embodiment, the data for the ‘normal’ growth curve could be obtained from at least one but potentially more than one cell culture chamber (acting as ‘control’) while the data for the treated spheroids could be obtained from at least one other cell culture chamber but potentially more than one (acting as ‘test’) grown in parallel with control cell culture chamber(s) (ideally at the same time and possibly inside the same incubator unit). This would achieve higher reproducibility and experimental accuracy than can currently be achieved when experiments are carried out in a stepwise manner or in different instruments. Additionally, data from one or more than one group of cell culture chamber devices can be automatically compared against each other to follow the effects of a treatment of at least one of the groups (as described above). Thus, the incubator system can provide advanced data analysis using the data available from image analysis in a non-invasive manner.

In some embodiments, the one or more processing units is configured to extract or derive data from the one or more monitoring signals and to sort it into different categories based on one or more characteristics of the extracted or derived data, wherein at least one of these predetermined categories correspond to cells or cell clusters, and to provide data about cell proliferation over time. In some further embodiments, the information about cell proliferation is visualized to a user.

According to further aspects and/or further embodiments is provided one or more processing units (e.g. of an incubator and/or a computer as disclosed herein) configured to carry out one or more of the image analysis methods or steps thereof as disclosed herein. In some embodiments, the incubator(s) and/or the user interface device is further configured to perform data logging and/or documentation e.g. collecting and storing data such as rotational speed, biomass (DNA protein, number of cells), the rate of growth, the size and standard deviation (reproducibility), and any sensor output, each measurement being made over time and e.g. including averages as well as duration and number of pauses (without rotation), etc. for at least some, e.g. all, of the cell culture chamber devices. Data instituted modifications of incubator function, and the time at which they were executed, will at least in some cases be recorded as will any notifications sent to the user. This may e.g. be supplemented with video(s) and/or still image(s) and with data from image analysis. The data of the data logging or documentation may e.g. be stored (e.g. also) in a cloud computing environment.

A user interface device may for example be configured for online monitoring of the signals obtained by the monitoring device(s) of the incubator, storage of such signals, image analysis and the output of any image analysis performed (e.g. one or more as illustrated in any of FIGS. 7A-7D).

In some embodiments, the incubator is further configured to receive, via the network, user input control data obtained by a user interface device and/or another external computational device (e.g. client, server, master, etc.), and to change or adapt operation in response to at least a part of the received user input control data.

In some further embodiments, the incubator (e.g. a master unit) is configured to receive further user input control data and communicate, at least a part thereof, to another incubator (e.g. a slave unit), wherein the other incubator is configured to change or adapt operation in response to at least a part received further user input control data.

### Definitions

All headings and sub-headings are used herein for convenience only and should not be constructed as limiting the invention in any way.

The term “cell culture” herein refers to the maintenance in the living state of any kind of cells, cell clusters, tissue-like structures, tissue biopsies, spheriods, organoids, or similar samples obtained or initially cultured by any method known in the art.

The term “cells” herein refers to primary, immortal or stem cells (including pluripotent or induced (in any way) pluripotent) or genetically modified cells from any type of living organism, whether archaea, prokaryote or eukaryote, and also includes viruses or other entities that need living cells to replicate.

The term “image processing” is often (but not always) applied to the manipulation of whole images to obtain an enhanced image (e.g. adjusting the contrast or brightness or rotating it) whereas the term “image analysis” is often (but not always) applied to the extraction of meaningful information from images (for example the identification of parts, features or elements of an image such as the identification of a tree in an image of a landscape). The two terms (image processing and image analysis) are primarily used herein according to these definitions but may also be used interchangeably herein.

The term “monitoring device” is any device that can record or detect one or more characteristic about the cell culture chamber or the cell culture media or its content at any time during the operation of the incubator system

A “monitoring signal” is any signal which results from the use of the monitoring device including but not limited to registration, visualization, video, images, frequencies, intensities, and spectroscopic data. In at least some embodiments, the monitoring signal comprises or represent digital image or digital video.

The use of any and all examples, or exemplary language provided herein, is intended merely to better illuminate the invention and does not pose a limitation on the scope of the invention unless otherwise claimed. No language in the specification should be construed as indicating any non-claimed element as essential to the practice of the invention.

This invention includes all modifications and equivalents of the subject matter recited in the claims appended hereto as permitted by applicable law.

### BRIEF DESCRIPTION OF THE DRAWINGS

FIG. 1 illustrates a perspective view of an example of a bioreactor used for culturing cells in three dimensions (3D).

FIG. 2 schematically illustrates a schematic perspective side view of an exemplary embodiment of alternative illumination paths in an incubator system (front- and back-illumination) as disclosed herein.

FIG. 3 schematically illustrates a plan first (e.g. front) view image of a cell culture chamber device.

FIG. 4 schematically illustrates the distribution of equally sized spheroids at 5 different speeds of rotation of the cell culture chamber.

FIG. 5 schematically illustrates one solution to the division of the image of a cell culture chamber into zones which can be used by image analysis for the regulation of the speed of rotation of the cell culture chamber device.

FIG. 6 schematically illustrates another solution to the division of the image of a cell culture chamber into zones which can be used by image analysis for the regulation of the speed of rotation of the cell culture chamber device.

FIGS. 7A-7D schematically illustrate a computer program flowchart implementing respective exemplary embodiments of some of the functions described herein.

### DETAILED DESCRIPTION

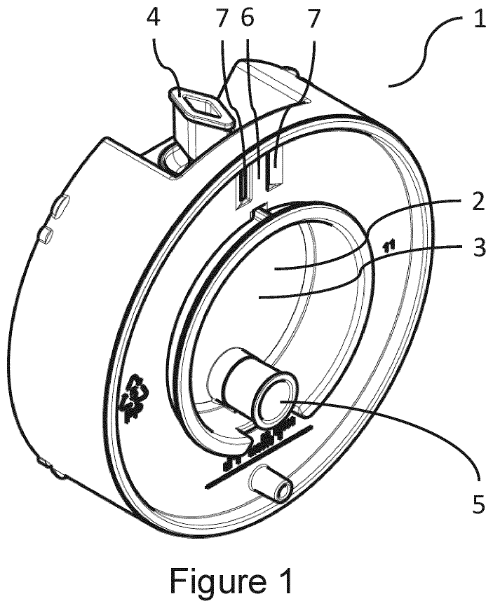

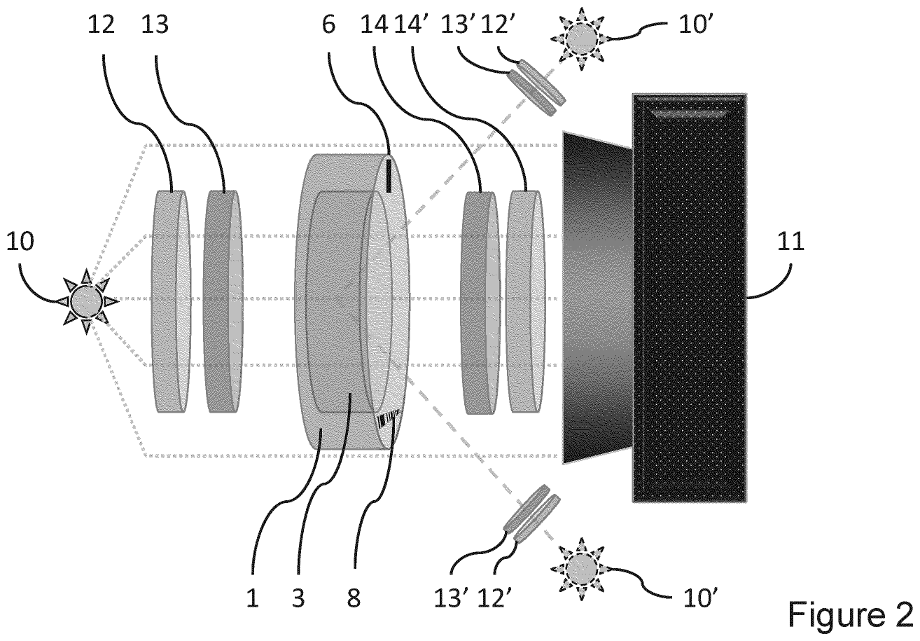

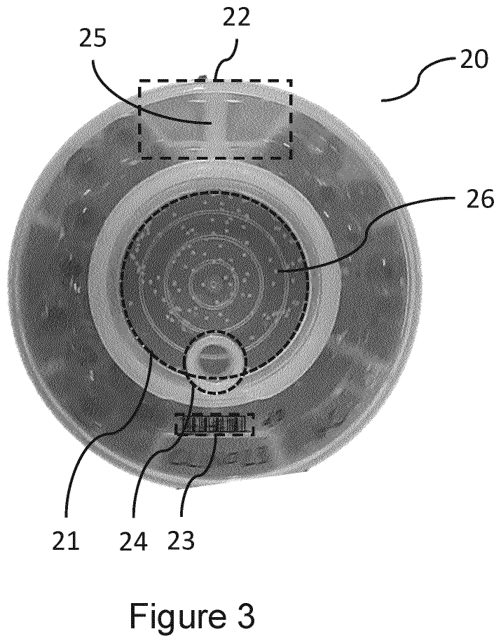

FIG. 1 schematically shows a perspective view of one example of a cell culture chamber device 1, illustrating some of the elements that can be found in such a device. Specifically, 2 indicates a substantially transparent primary viewing end or part. Behind this part is the enclosure for cell culture 3. 4 is a plug which locates into a circumferential port allowing access into the enclosure. 5 is a plug which locates into a front port allowing access into the enclosure. 6 is an element of the cell culture chamber device which can be used as a fiducial marker. To each side of this, there is a gas exchange port 7 (with an inlet and an outlet). The inlet and the outlet of the gas exchange port 7 defines or promotes the element 6 thereby making it suitable for a fiducial marker. A bar code (such as 8 in FIG. 2 or 23 in FIG. 3) or similar can also be used as a fiducial marker.

FIG. 2 schematically illustrates a schematic perspective side view of an exemplary embodiment of two different illumination paths in an incubator system as disclosed herein. 10 and 10′ indicate back- and front-illumination sources, respectively, and the dotted and dashed lines respectively illustrate the resultant light paths. 1 is a cell culture chamber device as disclosed herein and 3 is a cell culture chamber (enclosure) thereof in which cells or cell clusters are cultured. 11 indicates a monitoring device. 12, 12′, 13, 13′, 14 and 14′ represent one or more optional filters or lenses to selectively include or exclude particular wavelengths of light or lead the light to and from the cell culture chamber device 1. 6 is a fiducial marker (e.g. or preferably as shown in FIG. 1) and 8 is a bar code or similar/other identifier, which can be used for identifying a particular cell culture chamber device when placed in an incubator system comprising a plurality of such cell culture chamber devices and/or across several incubator systems.

FIG. 3 illustrates a plan first (e.g. front) image 20 of a cell culture chamber device 1 schematically shown in FIG. 1. The area enclosed by the dotted lines 21 is the part of the image corresponding to the cell culture chamber 3. 22 and 23 are the parts of the image corresponding to the fiducial mark 6 and the bar code 8, respectively. 24 and 25 are the part of the image corresponding to the front and circumferential ports (and plugs), respectively. The light spots (e.g. 26) within 21 are spheroids (here seen with front-illumination). FIG. 3 also illustrate a number of moisturising elements (seen as spheres circumferentially outside the area 21/the cell culture chamber 3—here in the form of water beads. In at least some embodiments, the moisturising elements is/are configured to humidify or moisturise air or gas in the vicinity of or being adjacent to at least a part of a gas exchange interface or a circumferential gas permeable membrane in contact with the cell culture chamber 3.

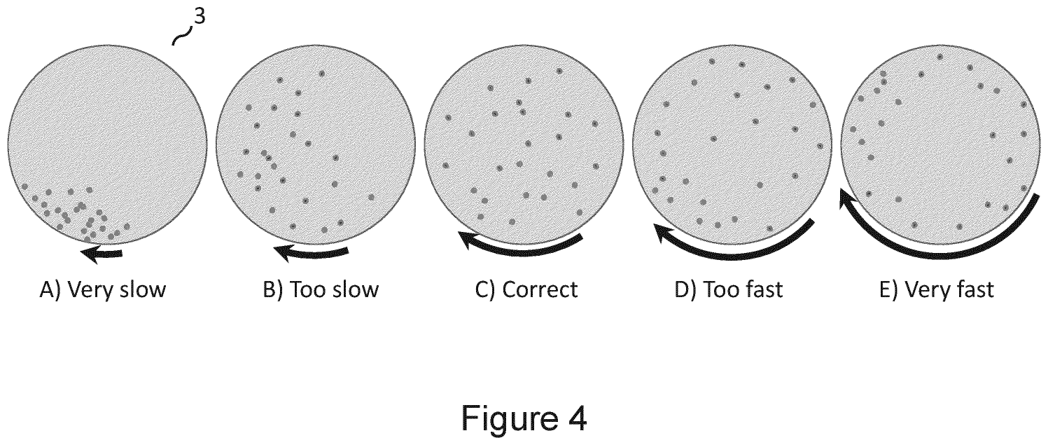

FIG. 4 schematically illustrates five exemplary images of a typical distribution of cell clusters or spheroids in the cell culture device enclosure 3 rotating at five different speeds from very slow, through too slow, correct, to too fast and very fast. The arrow illustrates the speed and direction of rotation. Note the different distribution of the spheroids in the different images (of FIGS. 4A to 4E). For the sake of clarity, many details, for example the front port, have been excluded.

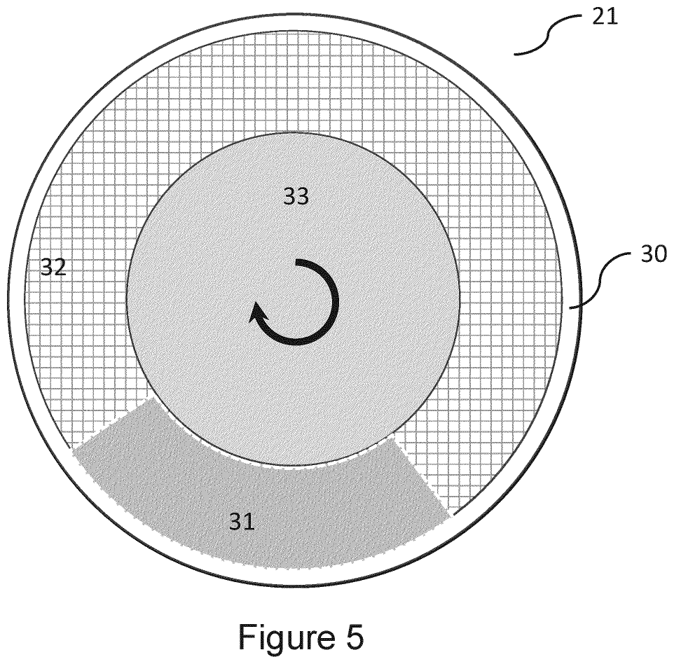

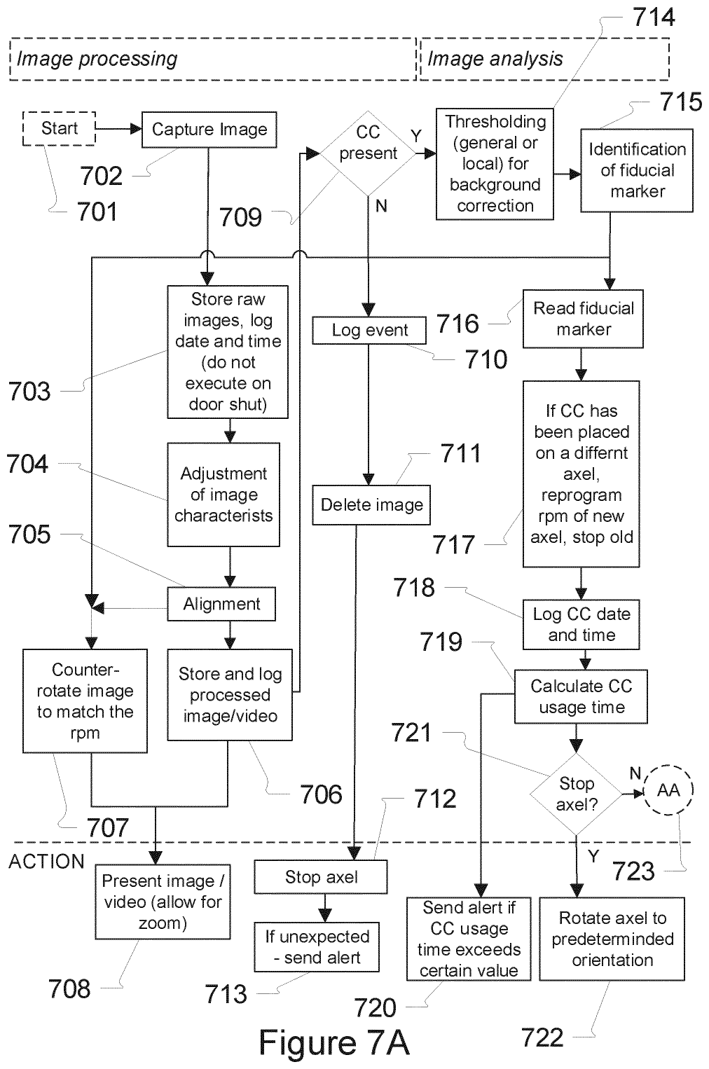

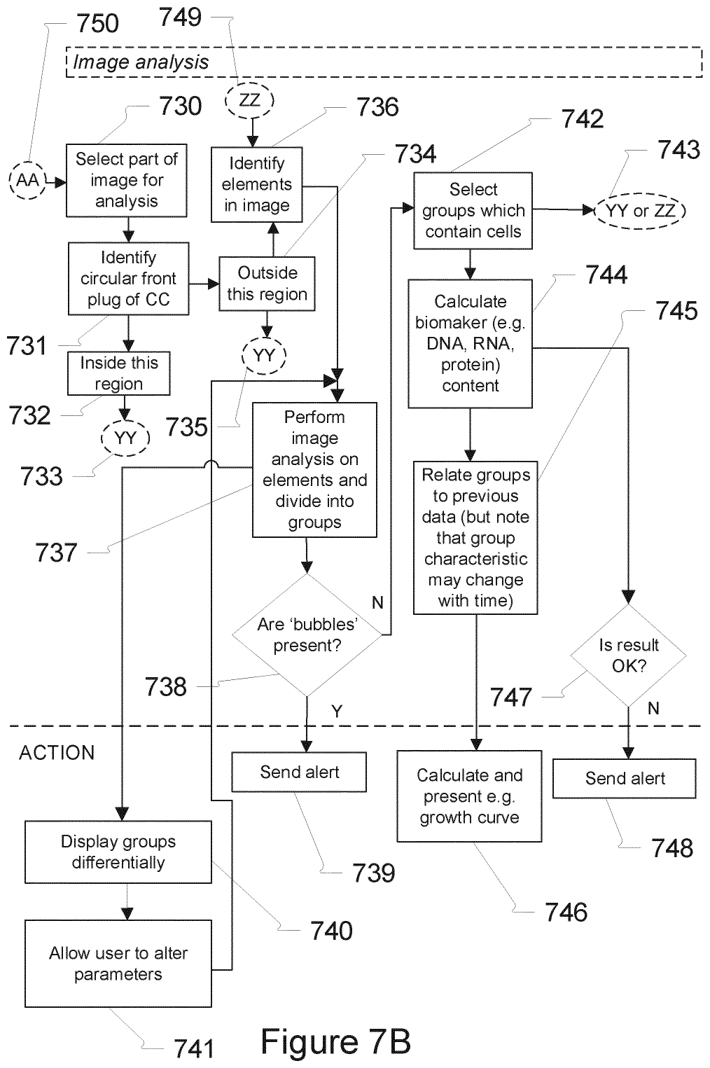

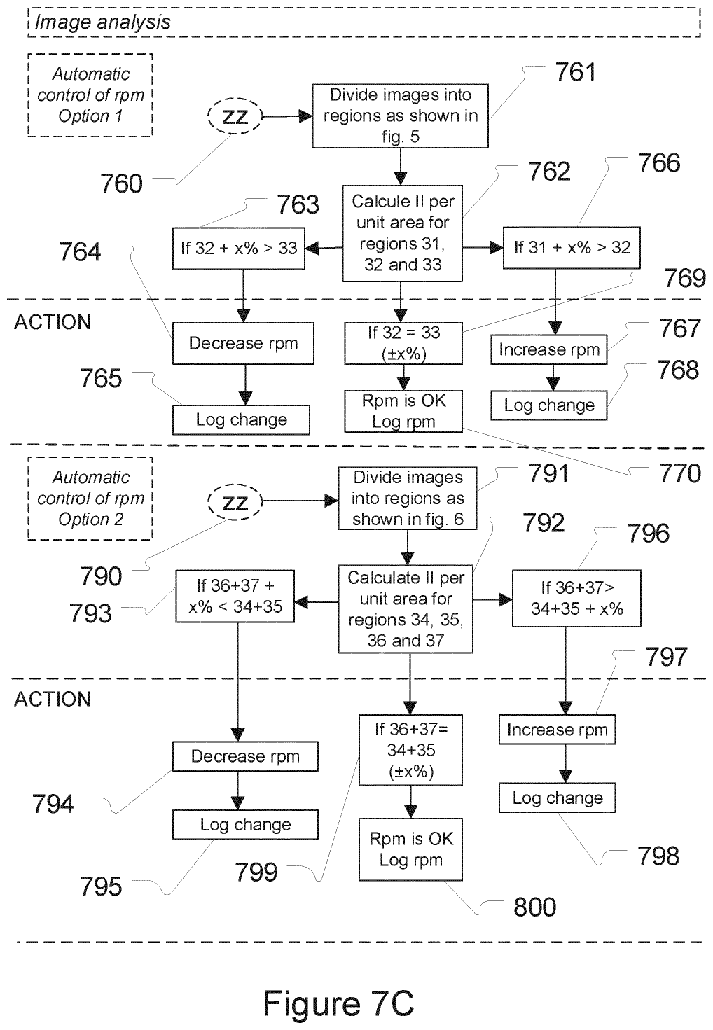

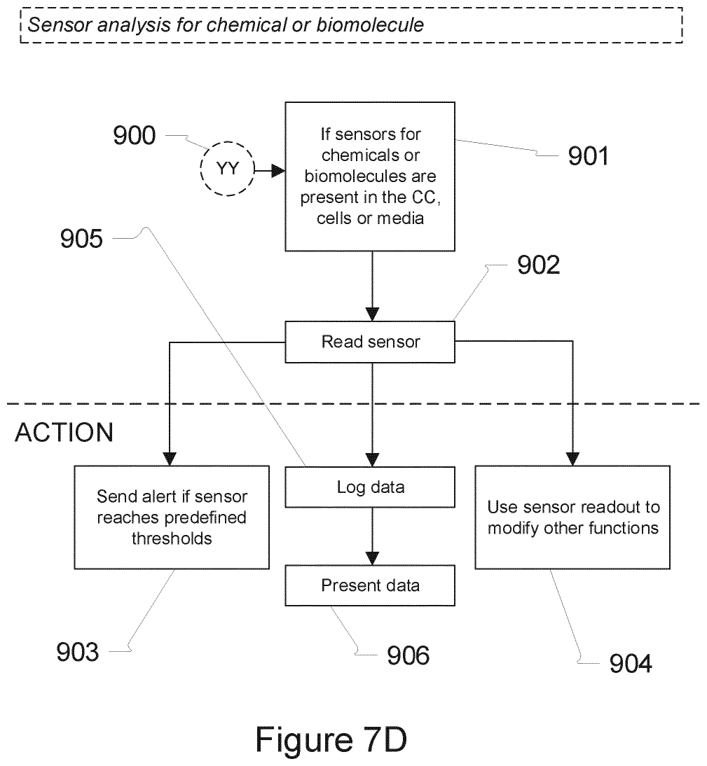

FIG. 5 schematically illustrates zones, areas, parts, segments, or the like of an image of the cell culture chamber and indicative examples of different zones, etc. respectively marked 30, 31, 32 and 33 used for image analysis according to different embodiments, e.g. or preferably as disclosed in connection with FIGS. 7A-7D (in particular FIG. 7C). The actual sizes and distribution of these zones are shown for illustration and many other combinations and shapes can be used. The arrow illustrates the direction of rotation. Zone 30 is a relatively narrow zone where potential edge effects (for example optical aberrations and reflections at the edge of the chamber) occur and for this reason may be excluded from image analysis. In one example of an embodiment (see e.g. Option 1 of FIG. 7C) which regulates the speed of rotation of the cell culture chamber device, zones 31 and (32+33) are used for image analysis when rpm is slower than optimal and zones (31+32) and 33 are used for image analysis when rpm is faster than optimal. Zone 31 may also be referred herein as a third region, zone 32 may be referred to herein as a first region, and zone 33 may be referred herein as a second region.

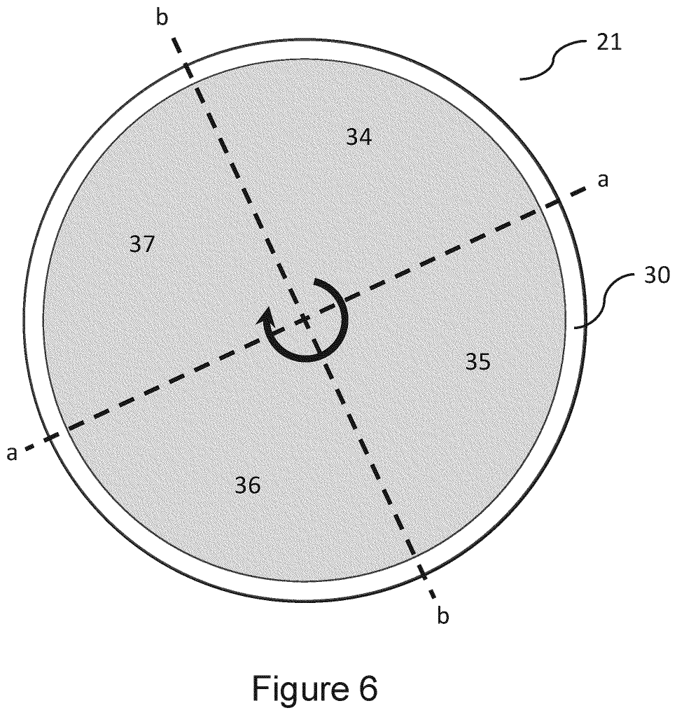

FIG. 6 schematically illustrates a plan first (e.g. front) view image of the cell culture chamber and indicative examples of zones marked 30, 34, 35, 36 and 37 used for image analysis according to different embodiments, e.g. or preferably as disclosed in connection with FIGS. 7A-7D (in particular FIG. 7C). The arrow illustrates the direction of rotation. The dashed lines (a - - a and b - - b) illustrate or define different zones, areas, parts, segments, or the like of the image of the cell culture chamber according to another embodiment (see e.g. Option 2 of FIG. 7C) of regulating the speed of rotation of the cell culture chamber device. In this example, zones (36+37) are compared with zones (34+35) to regulate the speed of rpm. In an alternative embodiment, 36 can be compared to 34. a - - a is typically at right angles to b - - b. The degree to which a - - a is displaced from the vertical (in the direction or rotation) will typically depend on the speed of rotation, the viscosity of the medium and the size of the spheroids. Zone 34 may also be referred herein as a sixth region, zone 35 may also be referred herein as a seventh region, zone 36 may also be referred herein as a fifth region, and zone 37 may also be referred herein as a fourth region.

The zones shown in FIGS. 5 and 6 relate to different regions in the image analysis and not to any physical zones in the cell culture chamber itself. In other words the zones can be considered as ‘cutting templates’ used to divide different regions of the image of the cell culture device. As such the zone template does not rotate (with the rotated cell culture chamber device). A particular zone template depends on the specific physical design of the used cell culture chamber device (as seen from the front/the side being monitored by a monitoring device).

FIGS. 7A to 7D schematically illustrates a flowchart overview of a computer implemented program or method executing respective various embodiments of image processing and/or image analysis functions as described herein (including regulation of the speed of rotation of the cell culture chamber device and the use of sensors to extract further information). The image(s) to be processed and/or analysed will typically be obtained or provided by one or more monitoring devices as disclosed herein such as one or more imaging or vision systems or devices such as one or more cameras, CCDs, etc. According to the illustrated flowchart, the program ‘start’s 701 for a particular cell culture chamber device (e.g. or preferably among a plurality) when the device in question is mounted on an axel of a suitable incubator system or similar and ‘stops’ when the device is removed again from the axel or the program otherwise is stopped. The lower ‘ACTION’ box (light grey bottom area of the Figures) illustrates where steps of the computer implemented program can modify the operation of the incubator system itself and/or sends data/information or alerts to one or more users.

The relevant part of the computer implemented program can be carried out on single images or images from a video sequence, whereas certain functions typically will require multiple, possibly sequential images. These images may be ‘raw’ or processed in some way to increase processing speed (e.g. compressed).

Illustrated, is an example of a possible set of steps and they may not need to occur in the sequence presented. Additionally, not all steps need to occur, and some may need to be repeated. A variety of different algorithms are known for some of the individual steps (e.g. background correction or element identification) and many of these can be used to carry out the desired image processing or image analysis task.

Depending on the task, illumination can be from the front (same side as the monitoring device/camera) or back (e.g. so that contained spheroids are seen in silhouette). Illumination (especially for analysis using the sensors) may require switching (manually or automatically) between different colours and/or wavelengths and/or filters.

In at least some embodiments, a user is able to pre-program certain operations (e.g. collect images every 6 hrs or at another interval). Some operations will typically have default starting values—e.g. rpm(start) could be 14.0 rpm and it will typically be possible for a user to change these default values.

In at least some embodiments, there are other ways to regulate the rpm. For example, a user could input manually what rpm should be employed. Alternatively or in addition, the flowchart comprises one or more steps using a location or trajectories of cells or spheroids. Such steps are not illustrated in the flowchart.

Abbreviations used in the flowchart are: CC: cell culture chamber device; II: integrated intensity (sum of pixel intensity values (before or after background subtraction) within a defined area of the image; rpm: revolutions per minute; Y and N: yes and no responses to decision points (diamond boxes) (where no Y or N is given, it is assumed that this response does not lead to a particular action); and Double letters in dashed ovals (e.g. the dashed oval around AA) illustrate where parts of the program connect to another part.

FIG. 7A schematically illustrates a (part of a) flowchart implementing respective exemplary embodiments of some of the functions described herein.

The a computer implemented program or method initiates at step 701 and proceeds to step 702 where at least one image is obtained or captured by one or more imaging or vision systems or devices as disclosed herein. As mentioned, a single image, a number of subsequent images, or a video sequence may be obtained depending on use/embodiment. At step 703, the obtained image(s)/video is stored (or one or more representative versions thereof) and an associated time and date (e.g. together with other relevant data/information) is logged. Shutting the incubator door initiates a subroutine which checks which CC are present on which axels but does not necessarily cause the collection of an image (702) and subsequent processing (703-706) (apart from determining whether a CC is present or not for one or more axels).

Step 703 proceeds to step 704 where—at least in some embodiments and as illustrated—adjustment of one or more image characteristics are carried out. After storing a raw image in 703, the image may be processed to enhance subsequent analysis. This can include, but is not limited to focussing, noise reduction, smoothing, adjusting the brightness and contrast (possibly in a non-linear manner). Next, step 705 is executed where an alignment is carried out, in which the image is aligning (sliding the image in the X and Y dimensions) or centring the image on a particular point (e.g. the centre of rotation of the CC, before optionally proceeding to step 707 where one or more of the obtained/captured images (e.g. of a video) is counter-rotated to match (or counter) a current rpm of the axel or CC. In this way, the image data is rotated so that the content of the image data may be displayed or processed as it appears or is stationary (despite being rotated according to the current rpm). Accordingly, the image data is ongoingly or intermittently processed in such a way that each image, or part of such (e.g. the part corresponding to the cell culture chamber device), is rotated ‘backwards’ by an amount corresponding to the ‘forward’ rotation occurring during the time between taking one image frame and the next. This could e.g. be done (in particular if proceeding to step 707 from step 705) simply by obtaining data or a value representing a current rpm of rotation of a contained CC (which often will be known) and adjust the image/derive the counter-rotation using this. Alternatively/additionally, this could e.g. be performed (in particular if proceeding to step 707 from step 715; see also later) by locating a bar code and/or a fiducial marker and keeping its/their position constant or fixed in the counter-rotated images. As already mentioned, when the spheroids are in ‘stationary orbit’ relative to the cell culture chamber device, this would facilitate the observation of individual spheroids because they would appear to remain roughly motionless in the image, enabling a closer inspection. Maintaining in effect the spheroids essentially motionless would permit tracking of individual spheroids over extended periods of time and permit kinetic observation of biological processes in a single spheroid.

Step 705 proceeds in parallel (to the optional step 707) to step 706 where processed (by steps 704 and 705) versions of the obtained image(s)/video (or representative versions thereof) is stored and logged.

After step 706 and 707 have been carried out, step 708 is carried out presenting the processed image, images, or video (e.g. or preferably) allowing for zoom and/or other typical user image manipulating possibilities. The presentation may e.g. be on the incubator and/or on a connected user device that may be locally or remotely present.

It is to be understood that steps 702 to 708 may loop intermittently or more or less in real-time. If in real-time, then logging and storing only intermittently. These steps also mainly involve image processing as indicated in FIG. 7A. In some alternative embodiments (not illustrated), step 707 may also proceed to step 706 rather than to step 708 allowing for storing and logging of the counter-rotated image in addition or instead of the directly obtained image (otherwise rotating together with the CC).

Step 706 also branches out to step 709 where it is checked or tested whether a (e.g. particular) CC is present (on a respective axel or in the incubator) or not. In case of No, the method proceeds to step 710, where it is logged that the (respective) CC is not present, step 711 where the otherwise stored image(s)/video is deleted, step 712 where the axel (with no CC) is stopped, and step 713, where—if no CC present was unexpected—an alert is triggered or sent to one or more users and/or other systems/devices. Determining whether it (no CC present) was unexpected may e.g. be carried out in response to values or settings of the incubator and/or user-specified values or settings.

If the test 709 results in Yes, the method proceeds to step 714 and subsequent steps mainly involving image analysis as indicated. At step 714, a suitable (general or local) thresholding or similar is, at least in some embodiments, carried out for background correction. Proceeding to step 715, identification (using image analysis) of one or more fiducial and/or identification markers (in the processed image(s)/video) is carried out e.g. or preferably on the stored processed image(s) of step 706. At step 716, the one or more identified fiducial and/or identification markers are read or interpreted to obtain an associated identifier or similar of the CC that the fiducial and/or identification marker(s) is/are for, thereby enabling determination of which CC is present. At step 717, if the CC (as determined by the identified and read fiducial and/or identification marker(s)) is determined to be on a new/changed position (i.e. on a new/changed axel), the rpm setting of the axel the CC is present is adjusted to fit the proper rpm for the identified CC and—if necessary—the previous (where the CC were last registered as being located) is stopped. This allows seamless change of CC position/axel location—e.g. by inserting the CC back into the incubator on a different axel after inspection or use—by identifying the CC and adjusting the rpms of whatever axel it is now located on to be the right ones (as associated previously with the CC).

At step 718, the obtained CC identifier and time and data is logged. At step 719, the CC usage time (of the identified CC) is calculated or updated (e.g. accommodating for any pauses, removals from the incubator, etc.). If the usage time exceeds a predetermined value, step 720 triggers or sends an alert.

At step 721 it is tested whether the axel (that the identified CC is secured to) should stop or not. In case of yes, step 722—at least in some embodiments—rotates the axel to a predetermined orientation (e.g. using the one or more fiducial and/or identification marker(s)) and halts to rotation of the respective axel. In case of no, the method proceeds to step 730 of FIG. 7B for additional image analysis as indicated by connection points 723 and 750 (of FIG. 7B).

FIG. 7B schematically illustrates a (part of a) flowchart implementing respective exemplary embodiments of some of the functions described herein.

At step 730 (proceeding hereto from ‘No’ of step 721 of FIG. 7B), one or more parts of an image in question is selected for (further) image analysis. At step 731, the CC is identified in the image. The annular region of the CC, where the water beads or another moisturizing agent is/are located, can be selected and used to determine how much water is present in them. In addition the circular observation window of the CC is located. Within this observation window region, the (circular) front plug (see e.g. 5 of FIG. 1 or 24 of FIG. 3) of the CC in question is identified and the image is divided into regions inside the plug (the flat clear inner part of the image which is surrounded by the edges of the plug) and outside the plug (i.e. the remaining part of the circular observation window region) (see e.g. FIG. 3). A sensor may be located inside the plug. If this is the case, at step 732, the sensor(s) is interrogated as described in FIG. 7D. This may require a predetermined combination of colours, wavelengths, filters followed by measurements of the II. Sensors may be located outside the plug region and if this is the case step 734 will lead to a similar interrogation of the sensor(s).

In step 736, elements of the image are identified, some of which may be used (via link ZZ 749 to FIG. 7C) for the regulation of the speed of rotation of the CC. Elements are further identified in step 737 by edge detection, thresholding, filtering and sorting using algorithms well known for image analysis. Elements may need to be divided when they overlap by features like shape, circularity and water-shedding. Characteristics of each element are then determined (including, position, dimensions, area, circularity, shape, smoothness, II) and one or more of these features used to sort the elements into groups. Usual statistical tools can then be applied to these groups (e.g. to determine average, standard deviation, etc.). These groups can be displayed to the user—either for example by colour overlay of the elements in the image, or by graphical or tabular or other means 740. Optionally the user can interact with some of the parameters used in image analysis to modulate their function 741. The alteration of a parameter would result in a recalculation, reidentification and representation of the image elements.

One of the image element groups identified may correspond to bubbles (for example characterised by high circularity, dark edges and light centres (or the reverse in negative images)). If these are detected (in step 738) an alert is sent to the user at step 739. Another of the image element groups 742 may correspond to individual cells (characterised by for example size) and other group may correspond to clusters of cells (‘spheroids’ or ‘organoids’). It is possible, at step 744, to calculate certain biological information from the characteristics or from the readout of sensors located in or on the cells via connection point 743. This biological information may be the number of cells present, content of DNA, RNA, protein or other biomarkers. Areas of the image outside of all of these elements (but still within the CC observation window will typically correspond to the growth media for the cells. This too can contain sensors or indicators of for example pH (e.g. phenol red) and so image processing can determine the pH of the solution in which the cells are growing. The user may assign thresholds or set points for any of these groups, features or characteristics 747 at which the program should issue alerts 748. An example of this could be when the image analysis calculates that the total number of cells (cells+cell clusters) exceeds a value indicating an over-population of the CC. This might prompt the user to split the culture or change the media more often.

Data collected at different timepoints may result in the identification of groups with differing characteristics. For example the average area of the ‘cell cluster’ group may increase with time 745. This can be used to calculate the growth rate of the cells and present the data to the user 746.

FIG. 7C schematically illustrates two possible ways or embodiments of automatically controlling the rpm of the cell culture chamber device present on a particular axel. The program (or subroutine) in its wording refers to previous diagrams and divides the image of the CC observation window into the zones illustrated—as examples in FIGS. 5 and 6, respectively, and assumes that the direction of rotation is clockwise. To decrease ‘noise’, multiple images can be averaged or processed in another suitable way before calculating an integrated intensity. By adding a tolerance value or factor x % (e.g. as in steps 763, 766, 793, and 796 799), it becomes possible to define a speed range in which rpm is acceptable. If the program results in an action to increase or decrease the rpm (e.g. as in steps 764, 767, 794, and 797), the image analysis has to be repeated from steps 761 or 791 after a predetermined length of time (typically less than an hour and more typically about 10 mins (depending on the size of the clusters and the viscosity of the medium in which the cells are growing) to allow the movement of the cell clusters to adapt to the new rpm speed). If the program results in a conclusion that the rpm is OK at steps 770 or 800, then the speed check should be repeated at the next predetermined timepoint. The ‘speed check’ should be repeated at regular (e.g. or preferably user programmable) intervals—e.g. each 30 mins for the first 4 days of culture and each 6 hrs after that.

Both of the above options could be carried out on image elements defined to be 3D spheroids or organoids (i.e. giving two further options to the two shown in FIG. 7C). In other cases, another feature (e.g. the integrated area of the spheroid or organoid could be used). Trajectory data of the spheroids or organoids, defined from multiple images, may e.g. also be used to regulate the speed of rotation.

According to rpm control option 1, the illustrated program or subroutine is initiated from somewhere else (e.g. step 743 or 749 of FIG. 7B) at connection point 760 before proceeding to step 761, where an obtained image (e.g. averaged) is divided into—as an example—a predetermined number of regions (here three as an example) corresponding to the ones illustrated in FIG. 5 and normalised to a unit area. At step 762, an integrated intensity (II) (sum of pixel intensity values (before or after background subtraction) within each of the defined areas or regions 31, 32, and 33 (of FIG. 5) is determined. If the II of area or region 32 (i.e. of a first region 32), e.g. plus a certain tolerance value or factor denoted x %, is greater than (assuming front lighting or similar) the II of area or region 33 (i.e. of a second region 33) (as tested in step 763), the rpm is decreased appropriately at step 764 before registering the decrease of rpm in a log/as a log change at step 765. The degree of change in rpm speed can be calculated in numerous ways (some of which will be more suitable than others for different situations). These ways include: user defined; a default percentage of the rpm; deduced from the previous round of changes in rpm speed which was necessary to achieve an acceptable rpm. Successive reiterations of rpm speed modification typically will utilise decreasing sized steps, the size of the step depending on the degree of difference between for example 32+x % and 33 or between 36+37+x % and 34+35.

If the II per unit area of area or region 31 (i.e. of a third region 31), e.g. plus a certain tolerance value or factor denoted x %, is greater than (assuming front lighting or similar) the II per unit area of the area or region 32 (i.e. of the second region 32) (as tested in step 766), the rpm is increased appropriately at step 767 (in a similar manner as described for decreasing the rpm) before registering the increase of rpm in a log/as a log change at step 768.

If the II of area or region 32 (i.e. the first region) is equal to the II of area or region 33 (i.e. the second region) potentially within a certain threshold as indicted by the tolerance value or factor+/−x % (as tested in step 769), step 770 concludes that the current rpm is ok or at least adequate and logs the current rpm together with an indication of this.

According to rpm control option 2, the illustrated program or subroutine is initiated from somewhere else (e.g. step 743 or 749 of FIG. 7B) at connection point 790 before proceeding to step 791, where an obtained image (e.g. averaged) is divided into—as an example—a predetermined number (here four as an example) of regions corresponding to the ones illustrated in FIG. 6. At step 792, an integrated intensity (II) (sum of pixel intensity values (before or after background subtraction) within each of the defined areas or regions 34, 35, 36 and 36 (of FIG. 6) is determined. If the sum of the II of areas or regions 36 and 37 (i.e. of a fourth 37 and a fifth 36 region), e.g. plus a certain tolerance value or factor denoted x %, is less than (assuming front lighting or similar) the sum of the II of areas or regions 34 and 35 (i.e. of a sixth 34 and a seventh 35 region) (as tested in step 793), the rpm is decreased appropriately at step 794 (as described above) before registering the decrease of rpm in a log/as a log change at step 795.

If the sum of the II of areas or regions 36 and 37 (i.e. of the fourth 37 and the fifth 36 region) is greater than (assuming front lighting or similar) the sum of the II of the areas or regions 34 and 35 (i.e. of the sixth 34 and the seventh 35 region) e.g. plus a certain tolerance value or factor denoted x % (as tested in step 796), the rpm is increased appropriately at step 797 (as described above) before registering the increase of rpm in a log/as a log change at step 798.

If the sum of the II of areas or regions 36 and 37 (i.e. of the fourth 37 and the fifth 36 region) is equal to the sum of the II of areas or regions 34 and 35 (i.e. of the sixth 34 and the seventh 35 region) potentially within a certain threshold as indicted by the tolerance value or factor+/−x % (as tested in step 799), then step 800 concludes that the current rpm is ok or at least adequate and logs the current rpm together with an indication of this.

FIG. 7D schematically illustrates a use of exemplary sensors, e.g. or preferably as disclosed herein, for the analysis of chemicals or biologically important molecules. The use of sensors will typically require the selection of a suitable combination of colours, wavelengths, and/or filters, located around or in connection with the cell culture chamber device (see e.g. 12, 12′, 13, 13′, 14 and 14′ in FIG. 2). Sensor measurement times can e.g. be user programmable or depend on other measurements (e.g. calculated DNA or protein content of the spheroids). To decrease ‘noise’, multiple images can be averaged before calculating the sensor output. According to the illustrated embodiment of sensor analysis, the illustrated program or subroutine is initiated from somewhere else (e.g. step 733, 735, or 743 of FIG. 7B) at connection point 900 before proceeding to step 901. Step 901 proceeds to step 902 if one or more sensors for chemicals or biomolecules are present in one or more of the cell culture chamber device (CC), the contained cells, and the media, where one or more sensors is read to obtain one or more types of sensor output data. At least some of the obtained data (and/or processed or derived data obtained therefrom) is logged at step 905 and presented at step 906. Furthermore, step 903 triggers or sends an alert if at least one sensor output reaches a respective predetermined threshold. Additionally, step 904 use at least some of the obtained one or more sensor output (or and/or processed or derived data obtained therefrom) to modify, initiate, start, stop, etc. one or more other functions.

Some preferred embodiments have been shown in the foregoing, but it should be stressed that the invention is not limited to these but may be embodied in other ways within the subject matter defined in the following claims.

It should be emphasized that the term “comprises/comprising” when used in this specification is taken to specify the presence of stated features, elements, steps or components but does not preclude the presence or addition of one or more other features, elements, steps, components or groups thereof.

In the claims enumerating several features, some or all of these features may be embodied by one and the same feature, component or item. The mere fact that certain measures are recited in mutually different dependent claims or described in different embodiments does not indicate that a combination of these measures cannot be used to advantage.

In the claims, any reference signs placed between parentheses shall not be constructed as limiting the claim. The word “comprising” does not exclude the presence of elements or steps other than those listed in a claim. The word “a” or “an” preceding an element does not exclude the presence of a plurality of such elements.

The mere fact that certain measures are recited in mutually different dependent claims does not indicate that a combination of these measures cannot be used to an advantage.

It will be apparent to a person skilled in the art that the various embodiments of the invention as disclosed and/or elements thereof can be combined without departing from the scope of the invention as defined in the claims.

1. An incubator system configured to illuminate at least one or more cell culture chamber devices and to rotate them about a respective predetermined axis, each cell culture chamber device comprising an enclosure, the enclosure configured to contain a cell culture media, and at least one viewing area configured to allow inspection of at least a part of the cell culture media, the incubator system further comprising at least one illumination device and at least one monitoring device, the at least one monitoring device configured to provide one or more monitoring signals of at least part of an illuminated cell culture media or an illuminated cell culture chamber, illuminated, by at least one of the at least one illumination device, with a signal of electromagnetic radiation, preferably incoherent or coherent ultraviolet, visible, infrared, and/or near-infrared light of broad or narrow wavelength spectrum, and wherein the incubator system further comprises or is in connection with one or more processing units configured to extract and/or derive data from the one or more monitoring signals, said data representing one or more aspects of the cell culture chamber device itself and/or the cellular activity occurring within.

2. An incubator system according to claim 1, where at least one of the one or more monitoring signals comprises a video signal or one or more image signals or data.

3. The incubator system according to claim 1 or 2, wherein at least one or some of the cell culture chamber devices each further comprises one or more fiducial marks, bar codes, or similar marks, and wherein the one or more processing units is configured to recognise such marks using image analysis and to identify a respective cell culture chamber device in response thereto, and wherein the incubator system is configured to control a rotational position and/or a rotational speed of the respective cell culture chamber device, and/or to monitor the use of the respective cell culture chamber device.

4. The incubator system according to claims 1-3, wherein the one or more processing units is configured to maintain a video or one or more images of the video of a rotating cell culture chamber device in an apparently stationary position through the use of the fiducial marks or otherwise.

5. The incubator system according to any one of claims 1-4, wherein the one or more processing units is configured to maintain a video or one or more images of the video of a rotating cell culture chamber device in an apparently stationary position by counter-rotating by an amount determined in response to a rotation per minute or other rotational velocity value of the rotating cell culture chamber device.

6. The incubator system according to claim 1-5, wherein the one or more processing units is configured to extract or derive data from the one or more monitoring signals and to sort it into different categories based on one or more characteristics of the extracted or derived data, wherein at least one of these predetermined categories correspond to cells or cell clusters, and to provide data about cell proliferation over time.

7. The incubator system according to claim 6, wherein the information about cell proliferation is visualized to a user.

8. The incubator system according to claim 1-7, wherein the incubator system is further configured to regulate the speed of rotation of one or more cell culture chamber devices in response to extracted or derived data of the one or more monitoring signals.

9. The incubator system according to claim 1-8, wherein the data extracted and/or derived from the one or more monitoring signals is or comprises one or more digital images and/or a digital video obtained of or for a contained cell culture chamber device, and wherein the incubator system is further configured to regulate the speed of rotation of the contained cell culture chamber device by performing image analysis on the one or more digital images and/or a digital video or parts thereof.

10. The incubator system according to claim 9, wherein the image analysis is performed on an image comprising or being divided into a first region (32), a second region (33), and a third region (31), and wherein the incubator system is configured to rotate the contained cell culture chamber device about the respective predetermined axis in a clock-wise direction and to
decrease the speed of rotation of the contained cell culture chamber device if a sum of pixel intensity values per unit area, or other image intensity metric or similar for the first region (32), alone or together with an added predetermined positive tolerance value (x %), is greater than a sum of pixel intensity values per unit area, or other image intensity metric, for the second region (33);
increase the speed of rotation of the contained cell culture chamber device if a sum of pixel intensity values per unit area, or other image intensity metric or similar, for the third region (31), alone or together with an added predetermined positive tolerance value (x %), is greater than a sum of pixel intensity values per unit area, or other image intensity metric, for the first region (32); and/or
maintain the speed of rotation of the contained cell culture chamber device for any other case.

11. The incubator system according to claim 9, wherein the image analysis is performed on an image comprising or being divided into a fourth region (37), a fifth region (36), a sixth region (34), and a seventh region (35), and wherein the incubator system is configured to rotate the contained cell culture chamber device about the respective predetermined axis in a clock-wise direction and to
decrease the speed of rotation of the contained cell culture chamber device if a sum of pixel intensity values per unit area, or other image intensity metric or similar, for the fourth region (37) added together with a sum of pixel intensity values per unit area, or other image intensity metric or similar, for the fifth region (36), by themselves or added together with a predetermined positive tolerance value (x %), is less than a sum of pixel intensity values per unit area, or other image intensity metric or similar, for the sixth region (34) added together with a sum of pixel intensity values per unit area, or other image intensity metric or similar, for the seventh region (35),
increase the speed of rotation of the contained cell culture chamber device if a sum of pixel intensity values per unit area, or other image intensity metric or similar, for the fifth region (36) added together with a sum of pixel intensity values per unit area, or other image intensity metric or similar, for the fourth region (37), is greater than a sum of pixel intensity values per unit area, or other image intensity metric or similar, for the sixth region (34) added together with a sum of pixel intensity values per unit area, or other image intensity metric or similar, for the seventh region (35) by themselves or added together with a predetermined positive tolerance value (x %), and/or
maintain the speed of rotation of the contained cell culture chamber device for any other case.

12. The incubator system according to claim 1-11, wherein the incubator system comprises at least two axels or drive units, each configured to rotate a respectively connected or received cell culture chamber device step, wherein the incubator system is further configured to identify a particular cell culture chamber device connected or received on a particular axel or by a particular drive unit and adjust the rotational speed of the particular axel or the particular drive unit to a rotational speed associated with the connected or received particular cell culture chamber device.

13. The incubator system according to claim 1-12, wherein the processing units is configured to analyse the one or more monitoring signals of the cell culture media for cells or cell cluster elements in the cell culture chamber and estimate the number of cells or the amount of a defined biomolecule present.

14. The incubator system according to claim 13, where the biomolecule is a protein, DNA, RNA, or other biomarker.

15. The incubator system according to claim 1-14, further comprising at least one sensor incorporated into one or more of: the cell culture chamber devices, the cell culture media, and/or the cells present to provide data about a chemical or biological process, and wherein said sensor may require a particular combination of wavelengths of electromagnetic radiation and filters to increase the signal to noise ratio.

16. The incubator system according to claim 15 where the sensor is a pH indicator, a fluorescent biomarker, or a chemical or an enzyme system.

17. The incubator system according to any of the preceding claims, wherein the one or more processing units is configured to calculate a status of a cell culture process in one or more of the cell culture chambers by extracting or deriving data from the one or more monitoring signals from said one or more cell culture chamber device, wherein the incubator system is further configured to automatically modify or adjust the operation of the incubator based on the calculated status and/or to alert a user about the calculated status thereby allowing the user to manually adjust the operation of the incubator system.

18. The incubator system according to claim 17, wherein the alert to the user is visualised on a smart phone, a tablet, a computer, or in an SMS or other electronic message.

19. The incubator system according to any of the preceding claims, wherein each respective predetermined axis is a horizontal or a substantially horizontal axis.
